(19) United States
(12) Patent Application Publication
(10) Pub. No.: US 2025/0352117 A1
(43) Pub. Date: Nov. 20, 2025

(54) SYSTEMS AND METHODS FOR MANAGING A CARDIAC MONITORING DEVICE WITH A MONITORING INTERVAL TRACKER

(71) Applicant: MURJ, INC., Santa Cruz, CA (US)

(72) Inventors: Richard Todd BUTKA, Santa Cruz, CA (US); Christopher S. IRVING, Santa Cruz, CA (US); Patrick BEAULIEU, Santa Cruz, CA (US)

(21) Appl. No.: 19/281,427

(22) Filed: Jul. 25, 2025

Related U.S. Application Data
(63) Continuation of application No. 19/142,797, filed on Jan. 1, 0001.
(60) Provisional application No. 63/215,647, filed on Jun. 28, 2021.

(51) Int. Cl. A61B 5/33; G16H 10/60; G16H 15/00; G16H 40/63; G16H 40/67; G16H 50/30

(52) U.S. Cl. CPC A61B 5/33; G16H 10/60; G16H 15/00; G16H 40/63; G16H 40/67; G16H 50/30

(57) ABSTRACT

Systems and methods include a monitoring interval tracker for managing transmission data originating from the cardiac monitoring device. The monitoring interval tracker generates monitoring intervals corresponding to multiple, different patient care modalities (e.g., an in-office care modality, a remote monitoring care modality, and/or a heart failure care modality). Upon receiving transmission data, a docket report corresponding to the transmission data, and/or an approval of the docket report, the monitoring interval tracker determines whether to extend the monitoring interval, generate a date of service, and/or create a new monitoring interval. Multiple monitoring intervals may run concurrently to track transmission data, docket reports, and approval timestamps for the multiple, different patient care modalities. Results of the monitoring interval tracker may be outputted as an interactive monitoring interval visualizer and/or a transmission dashboard to improve patient care by preventing failures to follow up with patients and by improving billing accuracy and efficiency.

### CROSS-REFERENCE TO RELATED APPLICATION

The present application is a continuation of and claims priority to U.S. patent application Ser. No. 19/142,797 entitled “SYSTEMS AND METHODS FOR MANAGING A CARDIAC MONITORING DEVICE WITH A MONITORING INTERVAL TRACKER,” which is a 371 of International Patent Application No. PCT/US2022/073231 entitled “SYSTEMS AND METHODS FOR MANAGING A CARDIAC MONITORING DEVICE WITH A MONITORING INTERVAL TRACKER,” which claims priority to and claims the benefit to U.S. Provisional Patent Application No. 63/215,647 entitled “SYSTEMS AND METHODS FOR MANAGING A CARDIAC MONITORING DEVICE WITH A MONITORING INTERVAL TRACKER.” Each of these applications is incorporated by reference in its entirety herein.

### TECHNICAL FIELD

Aspects of the present disclosure relate to managing patient medical devices for one or more patients and more particularly to systems and methods for managing a cardiac monitoring device.

### BACKGROUND

Implantable medical devices are regularly used to treat and/or monitor a variety of medical conditions. For example, cardiac monitoring devices, such as cardiac implantable electronic devices (CIED). CEIDs may include, without limitation: pacemarkers (PMs), which prevent slow heart rates using low-energy electrical pulses; implantable cardioverter defibrillators (ICDs), which are used to detect abnormal heart arrhythmias and deliver lifesaving shocks to prevent sudden cardiac arrest; implantable loop recorders (ILRs) and implantable cardiac monitors (ICMs), which continuously monitor cardiac data and transmit data to the clinic as prescribed by a clinician and at the patient's discretion; and the like. Such CIEDs store and may periodically transmit information relating to the operation of the device outside the body for analysis, programming, and/or the like. More particularly, CIEDs store and transmit information for in-office or remote monitoring by a medical provider.

However, medical providers are often responsible for managing a large number of patients having a range of different types of devices and different types of patient care. Each type of device may have a different transmission frequency with which it reports cardiac monitoring information to a clinic. Some devices may transmit cardiac monitoring information more consistently than others. Additionally, each type of patient care may have unique requirements for monitoring the patient. For instance, some types of patient care (e.g., remote monitoring) may require quarterly transmissions from the cardiac monitoring device, and other types of patient care (e.g., in-office monitoring) may require annual visits. Moreover, every transmission may require a corresponding docket report and an approval of the docketing report before a date of service can be established for the transmission. The complexity of managing cardiac monitoring device transmissions is further compounded when a single cardiac monitoring device is used for multiple types of patient care (e.g., remote monitoring and heart failure monitoring). As such, tracking every transmission of every cardiac monitoring device, generating docketing reports for the transmissions, approving the docketing reports, and establishing dates of service for the transmissions in a timely manner and without letting patient care diminish is a significant burden for clinics providing these services.

It is with these observations in mind, among others, that various aspects of the present disclosure were conceived and developed.

### SUMMARY

Implementations described and claimed herein address the foregoing problems by providing systems and methods for patient device management.

Cardiac monitoring by a professional typically occurs over the life of a patient. Such monitoring is subject to reimbursement and care guidelines. Cardiac care can vary for different types of patient care. For instance, cardiac care involves four remote data checks per year for patients having a pacemaker (PM) or implantable cardioverter-defibrillator (ICD) (“remote monitoring patient care”), at least one in-office check per year for PM or ICD patients (“in-office patient care”), and eleven remote data checks per year for Implantable Cardiac Monitor (ICM) or Implantable Loop Recorder (ILR) patients (“heart failure patient care”). Healthcare insurance in the U.S. is generally consistent with these guidelines. However, while healthcare insurance reimbursements exist, they often do not cover the labor expense involved in retrieving, reviewing, and documenting device transmissions. With the rapid adoption of new ILRs and ICMs and the increasing volume of data, the estimated transmission burden of these guidelines could exceed 800 million cardiac monitoring transmissions over the next five years. Compliance with these guidelines improves patient outcomes and lowers healthcare system costs, reducing mortality by nearly 2.5 times for patients with PMs and reducing hospitalizations by over 65% in ICD patients. However, due to the burdens involved with these guidelines, it is estimated that less than 30% of patients are monitored in accordance with these guidelines.

In one implementation, a medical device management platform for managing a cardiac monitoring device may include a monitoring interval tracker to track transmission data from a cardiac monitoring device by associating the transmission data with one more monitoring intervals. The one or more monitoring intervals may correspond to one or more patient care modalities, such as heart failure patient care, remote-office patient care, and/or in-office patient care. The monitoring interval tracker may calculate monitoring interval extensions as needed and categorize the transmission data, docket reports for the transmission data, and approvals of docket reports according to the patient care modalities and monitoring interval extensions to improve the accuracy of date of service DOS calculations while minimizing failures to provide proper follow up attention to transmissions.

Other implementations are also described and recited herein. Further, while multiple implementations are disclosed, still other implementations of the presently disclosed technology will become apparent to those skilled in the art from the following detailed description, which shows and describes illustrative implementations of the presently disclosed technology. As will be realized, the presently disclosed technology is capable of modifications in various aspects, all without departing from the spirit and scope of the presently disclosed technology. Accordingly, the drawings and detailed description are to be regarded as illustrative in nature and not limiting.

### BRIEF DESCRIPTION OF THE DRAWING

FIG. 1 shows a block diagram example network environment, including a medical device manager running on a server or other computing device coupled with a network, for managing one or more cardiac implantable electronic devices.

FIG. 2 shows a block diagram of a medical device data communication system including an example medical device data platform.

FIG. 3 shows a block diagram of the example medical device data platform.

FIG. 4 shows block diagram of an example monitoring interval tracker interacting with one or more databases and a provider portal

FIG. 5 shows a timeline diagram of example operations performed by the monitoring interval tracker to manage patient medical devices.

FIG. 6 shows a timeline diagram of example operations performed by the monitoring interval tracker to manage patient medical devices.

FIG. 7 shows a timeline diagram of example operations performed by the monitoring interval tracker to manage patient medical devices.

FIG. 8 shows a timeline diagram of example operations performed by the monitoring interval tracker to manage patient medical devices.

FIG. 9 shows a timeline diagram of example operations performed by the monitoring interval tracker to manage patient medical devices.

FIG. 10 shows a timeline diagram of example operations performed by the monitoring interval tracker to manage patient medical devices.

FIG. 11 shows a timeline diagram of example operations performed by the monitoring interval tracker to manage patient medical devices.

FIG. 12 shows a flow chart of example operations performed by the monitoring interval tracker to manage patient medical devices.

FIG. 13 shows an example monitoring tracker visualizer user interface for managing patient medical devices.

FIG. 14 shows an example monitoring tracker visualizer user interface for managing patient medical devices.

FIG. 15 shows an example monitoring tracker visualizer user interface for managing patient medical devices.

FIG. 16 shows an example monitoring interval configuration user interface for managing patient medical devices.

FIG. 17 shows an example transmissions dashboard user interface for managing patient medical devices.

FIG. 18 shows an example transmissions dashboard user interface for managing patient medical devices.

FIG. 19 shows an example billing report dashboard user interface for managing patient medical devices.

FIG. 20 shows an example computing system that may implement various systems and methods discussed herein.

### DETAILED DESCRIPTION

Aspects of the present disclosure involve systems, methods, computer program products, and the like for a medical device management platform for managing one or more implantable or otherwise wearable electronic devices. The electronic devices, may include medical devices, such as one or more cardiac monitoring devices (e.g., cardiac implantable electronic devices (CIEDs)). The medical device management platform may include a monitoring interval tracker which, in general, receives operational transmissions from one or more medical devices, such as CIEDs, of patients to manage the care of those patients, including receipt of data, reports, and information from such devices to enable a provider to view, document, report on, and generate a care strategy for the patients. Such operation transmissions may be received as transmission data at the monitoring interval tracker through many sources, including the corresponding device, a device programming machine, reporting from the patient or a manufacturer, or from a third-party entity receiving the transmissions from the device. Upon receiving the transmission data, the monitoring interval tracker may associate the transmission data with a corresponding monitoring interval to ensure that it receives a timely docket report, approval of the docket report, and DOS.

For instance, the monitoring interval tracker may generate a monitoring interval associated with the cardiac monitoring device, either prior to receiving the transmission data as part of an enrollment procedure or in response to receiving the transmission data. The monitoring interval may have a length of time based on a patient care modality rule, accessed from a rules database, corresponding to a particular type of patient care (e.g., remote monitoring care, in-office care, and/or heart failure care). The transmission data may be timestamped to indicate a date and/or time that the clinic received the transmission data (i.e., a “received date”). The monitoring interval tracker may determine whether the received date is within the monitoring interval by calculating whether the received date is prior to or later than an end date of the monitoring interval. When the received date is later than the end date, the monitoring interval tracker may access an interval extension rule from the rules database that indicates how to adjust the monitoring interval to capture the later received transmission data. For instance, if the end date occurs and still no transmission data has been received for the monitoring interval, the monitoring interval tracker may access a first interval extension rule indicating that the monitoring interval is to be extended on a day-by-day basis, creating an extended end date for the monitoring interval each day until the transmission data is received. When the transmission data is received during an extended monitoring interval, the extended monitoring interval may end and, in some instances, a date of service (DOS) may be generated for the extended monitoring interval on the received date (which may also be an extended end date). Additionally or alternatively, the monitoring interval tracker may access a second interval extension rule from the rules database indicating that, upon reaching the end date and without receiving the transmission data prior to the end date, the monitoring interval (e.g., a first monitoring interval) is to be closed and a second monitoring interval (e.g., having a same length of time as the first monitoring interval, for instance, as instructed by the patient care modality rule) may be generated with a new, second end date.

In some examples, the monitoring interval tracker may determine whether DOS criteria is met in order to generate the DOS. For instance, the DOS criteria may include the received date of the transmission data being prior to the end date (or the extended end date) of the monitoring interval, a docket report being generated for the transmission data prior to the end date (or the extended end date); and receiving an approval timestamp for the docket report indicating an approval date that is after the received date and after a docket report date, but before the end date (or extended end date). The monitoring interval tracker may access a plurality of rules from the rules database, such as one or more patient care modality rules and/or interval extension rules for generating and extending the monitoring intervals, according to the type of patient care, to ensure that the DOS criteria is met. For example, the monitoring interval tracker may receive the transmission data prior to the end date, and may generate a docket report prior to the end date, but may still be yet to receive the approval timestamp indicating an approval date for the docket report upon an occurrence of the end date. As such, the monitoring interval tracker may access a third interval extension rule from the rules database indicating that the monitoring interval is to be extended day-by-day, creating an extended end date each day, until the approval timestamp is received, satisfying the DOS criteria for the monitoring interval.

In some examples, the monitoring interval tracker may generate multiple, concurrently running monitoring intervals for the cardiac monitoring device, each of the concurrently running monitoring intervals corresponding to a different patient care modality rule and, as such, having different lengths of time and different end dates. Multiple docket reports may be received or generated for the transmission data, such as a docket report for each of the concurrently running monitoring intervals. In some instances, the monitoring interval tracker may receive a first approval timestamp for a first docket report of the multiple docket reports before the end date, on the end date, or on the extended end date, while still lacking a second approval timestamp for a second docket report of the multiple docket reports. For instance, the first docket report for a first monitoring interval for heart failure patient care may be approved within the first monitoring interval, while a second docket report for a second monitoring interval for remote monitoring patient care remains unapproved. The monitoring interval tracker may generate the DOS for the transmission data for the approved docket report even though the second docket report remains unapproved. Moreover, the monitoring interval tracker may receive the second approval timestamp for the second docket report during a third monitoring interval (e.g., generated subsequent to the first monitoring interval). Yet the monitoring interval tracker may recognize that the second approval timestamp corresponds to transmission data that has already received a DOS (based on the first approval timestamp for the first docket report), and omit generating a second DOS based on the second approval timestamp.

In some examples, the medical device management platform may include interface tools such as a monitoring interval visualizer for presenting information generated by the monitoring interval tracker on a display of a clinic providing the patient care, via a graphical user interface (GUI). For instance, the monitoring interval visualizer may present monitoring intervals as interactive interval shapes (e.g., bars, blocks, lines, etc.) on one or more timelines. The interactive interval shapes may include indicators or icons corresponding to the received date, a docket report status, the docket report date, and/or the approval date. Upon receiving user input, the interactive interval shapes and/or indicators may present various transmission related data to a permitted user, such as the received date of the transmission data, the docket report date, the approval date, the docket report (including a name of a medical personnel that prepared the docket report and/or medical personnel that approved the docket report), and/or one or more action needed indicators corresponding to the monitoring intervals. In some examples, the interface tools may include a transmissions dashboard to aggregate transmission related data for a particular clinic from a transmission database, generate one or more transmission metrics for the particular clinic, and present the transmission metrics on a display of the particular clinic. Statistics and other measurements of the received transmissions and resulting care protocols may also be provided through the interface tools to improve the operation and efficiency of a clinic, or to improve care to the patient, or to share data with another entity with access to the interface tools. In this manner, management of device transmissions is simplified and improved for users of the device management platform interface.

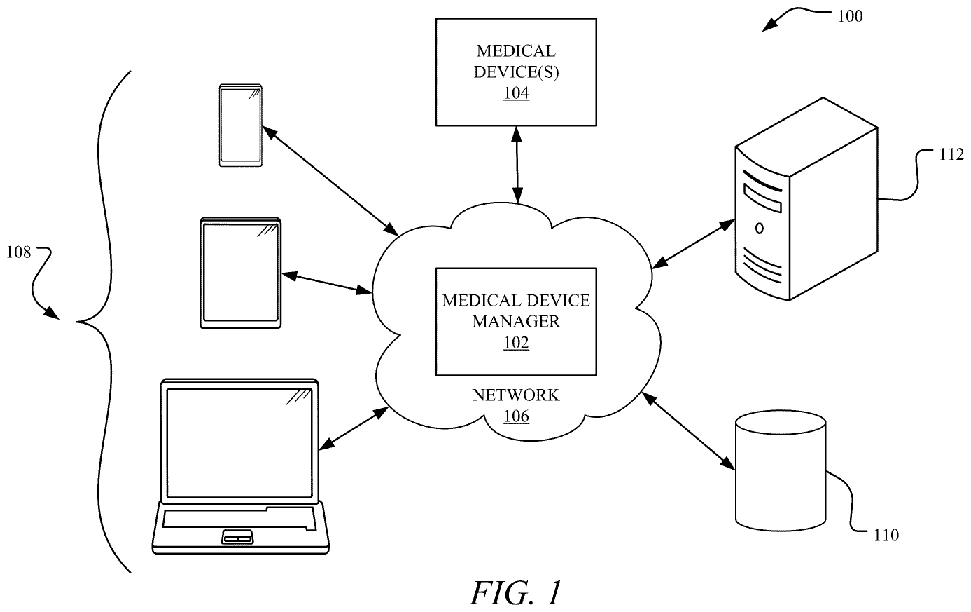

Reference is now made to FIG. 1 which illustrates an example network environment 100, including a medical device manager 102 running on a server 112 or other computing device coupled with a network 106, for managing one or more cardiac implantable electronic devices 104. In one implementation, a user, such as a member of a medical team and/or a third party to which access is delegated to manage or complete management services, accesses and interacts with a portal for the medical device manager 102 using a user device 108 to access or manage one or more medical devices via a network 106 (e.g., the Internet). The user device 108 is generally any form of computing device capable of interacting with the network 106, such as a personal computer, terminal, workstation, portable computer, mobile device, smartphone, tablet, multimedia console, etc. The network 106 is used by one or more computing or data storage devices (e.g., one or more databases 110 or other computing units described herein) for implementing the medical device manager 102 and other services, data structures, applications, or modules in the network environment 100.

In one implementation, the network environment 100 includes at least one server 112 hosting a website or an application that the user may visit to access the medical device manager 102 and/or other network components, including device programming machine that reside in a physician office and are utilized when in the presence of a patient. The server 112 may be a single server, a plurality of servers with each such server being a physical server or a virtual machine, or a collection of both physical servers and virtual machines. In another implementation, a cloud hosts one or more components of the network environment 100. The user devices 108, the server 112, and other resources connected to the network 106 may access one or more other servers to access to one or more websites, applications, web services interfaces, storage devices, computing devices, or the like that are used for integrated healthcare delivery. The server 112 may also host a search engine that the medical device manager 102 uses for accessing, searching for, and modifying patient data, team member data, education data, and other data. In one implementation, the medical device manager 102 provides access to data and/or other information of one or more medical devices 104 such as a cardiac monitoring device.

Medical devices 104 may be in communication with network 106 to provide operational data to the medical device manager 102. For example, and as described above, cardiac implantable electronic devices (CIED), such as implantable cardioverter defibrillators (ICDs) and the like, often transmit information relating to the operation of the device outside the body for analysis, programming, and/or the like. In some cases, a clinic retrieves data for the medical devices 104 from a device manufacturer secure website and/or from device programming machines physically located in a provider office. The data generated by the medical devices 104 is typically reviewed by a provider who then generates a docket report. A typical provider implants and monitors transmissions from the full range of manufacturers and device models and types. Providers rarely implant and monitor a single devices type. In terms of data transmissions and modalities of review of data transmissions, each of the manufacturers uses unique and proprietary data formats, displays, access protocols, reports, and programming machines. With the medical provider receiving data in this wide range of unique and disparate formats, it is challenging for the medical provider to efficiently manage the care of patients. The burdens of data access and management are a major impediment to better patient care. Cumbersome workflow creates excessive costs, impedes adoption of remote care, which is a superior care modality, leads to missed reimbursement opportunities, and forces some clinics to abandon remote care and instead push patients to the lower volume of in-office care, which leads to even further care reduction due to low patient compliance and greater healthcare system burdens associated with increased hospitalization. As such, as described in more detail below, the medical device manager 102 thus allows a user to manage, analyze, and/or store data from the one or more medical devices 104 across various device types and disparate manufacturers while improving DOS calculations and reducing follow up failures.

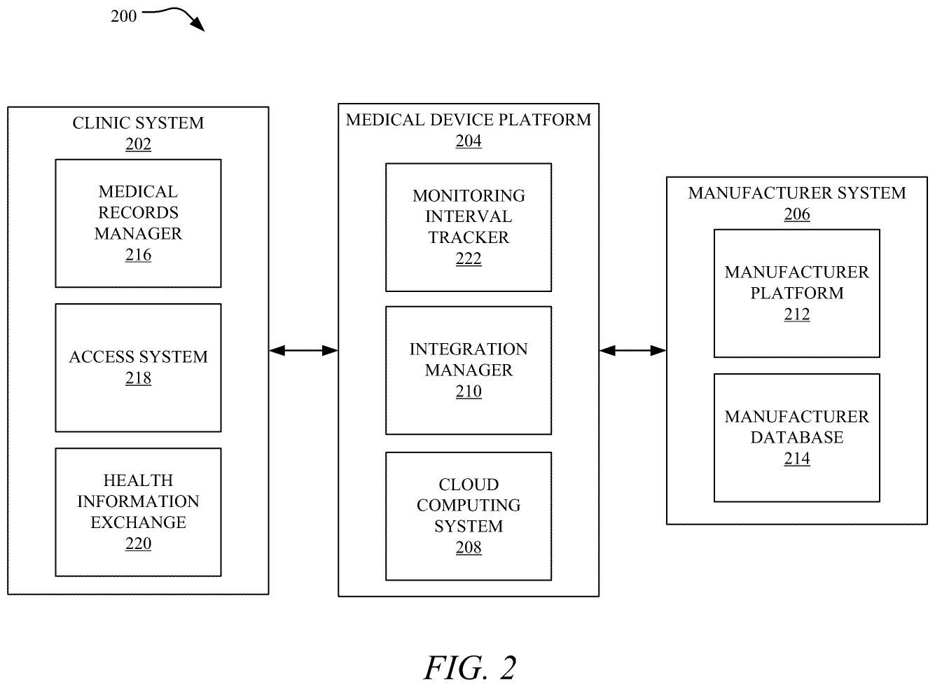

Turning to FIG. 2, the medical device manager 102 may include a medical device data communication system 200. In one implementation, the medical device data communication system 200 includes a medical device platform 204 in communication with multiple implantable medical device manufacturer systems 206 and multiple clinic systems 202. The medical device platform 204 may be communicatively coupled with the manufacturer systems 206 and the clinic systems 202 via a wide area network (WAN), such as, for example, the Internet. In some implementations, each of the manufacturer systems 206 may be associated with a unique implantable medical device manufacturer. In other examples, each of the manufacturer systems 206 may be associated with a unique pairing of an implantable medical device manufacturer and a particular medical clinic or office. In the example of the system 200 of FIG. 2, the manufacturer system 206 may include a manufacturer platform 212, such as, for example, a web server, that retrieves from a device database 214 diagnostic and related data previously uploaded from a plurality of implantable medical devices. Such data may have been retrieved previously from the various implantable medical devices by way of a clinic or office, or by way of a remote monitor, as described above.

The medical device platform 204, as depicted in FIG. 2, may include an integration manager 210 that accesses the diagnostic and related data for the implantable medical devices from each of the manufacturer systems 206. In an example, the integration manager 210 may provide a clinical information system (CIS) identifier associated with a particular clinic to the manufacturer platform 206 to retrieve the diagnostic data and related information, which may be in the form of Implantable Device Cardiac Observation (IDCO) messages. In some implementations, messages between the integration manager 210 and the manufacturer platform 212 may be in the form of alternative, enhanced, or augmented data messages. For example, IDCO messages often contain information formatted as summary reports in Portable Document Format (PDF). In other examples, IDCO-like messages may provide more detailed or “raw” data, such as numerical and/or graphical electrogram (EGM) data regarding arrhythmia or other cardiac episodes detected by the implantable device in integer, floating-point, or another data format. Such information may facilitate easier and/or more detailed processing of the device data within the medical device platform 204.

The integration manager 210 may then process the retrieved information to generate patient-oriented information and/or provider-oriented information. In some examples, the retrieved information from the various implantable medical devices may be processed into a format that is unified or generalized across all manufacturers and/or devices of a particular type. The integration manager 210 may then forward the processed information to a cloud computing system 208 that may provide one or more web portals for the clinic systems 202. In other examples, the integration manager 210 may forward the retrieved device data to the cloud computing system 208, which may then operate as an information processor to process the data. The cloud computing system 208 may also generate and provide analytics and other advanced information based on the processed information via the web portals. Examples of web portals and the generating and providing of analytics of the information are described in more detail below. The cloud computing system 208, in some examples, may include multiple computer devices or systems configured to perform the various operations ascribed herein to the cloud computing system 208.

As illustrated in FIG. 2, a user (e.g., one or more doctors, nurses, technicians, analysts, etc.) may employ a clinic system 202 to retrieve the processed device information, possibly including monitor interval tracking, the analytics, and other advanced information of one or more implantable medical devices. In some examples, the clinic system 202 may include a clinic access system 218 (e.g., a desktop computer, a laptop computer, a tablet computer, a smart phone, etc.) from which the user may access a web portal provided by the cloud computing system 208 to retrieve provider-oriented information, such as the medical device manager 102. As shown in FIG. 2, the clinic system 202 may also include one or more of an electronic medical records manager system 216 configured to store and facilitate access to medical records of the patients of the clinic, and a health information exchange (HIE) system 220 configured to exchange health and medical information for the patients of the clinic with other computing systems external to the clinic system 202. In one implementation, a user may sign on or log on to the cloud computing system 208, the medical recorder manager 216, and/or the HIE system 220 using a single sign-on (SSO) procedure, thus reducing the amount of time normally required by the user to access each of these systems individually.

In some examples, the integration manager 210 may retrieve the device diagnostic data and other device information via transmission data received by a communication connection with one or more of the implantable medical devices, thus possibly reducing the need for one or more of the manufacturer systems 206. In those examples and others, the integration manager 210 and/or the cloud computing system 208 may forward or “push” the unprocessed and/or processed device information, as well as the analytics and other advanced information to one or more of the manufacturer systems 206 for use by the corresponding manufacturers of the devices.

In some examples, the medical device platform 204 may include a monitoring interval tracker 222. The monitoring interval tracker may receive data (e.g., transmission data and transmission related data) from other components of the medical device manager 102, such as the integration manager 210. The monitoring interval tracker 222 may receive data from the clinic system 202, such as the access system 218. In some instances, the monitoring interval tracker 222 may categorize transmission data according to cardiac monitoring device from which the transmission data is received, a type of patient care (e.g., a “patient care modality”) associated with the transmission data (e.g., based on an association with the cardiac monitoring device or a patient being monitored by the cardiac monitoring device), and/or one or more monitoring intervals for the cardiac monitoring device. The monitoring interval tracker may access one or more rules from the database(s) 110 and perform calculations for generate extensions to the monitoring intervals and DOSs for the monitoring intervals. In some instances, the monitoring interval tracker 222 may perform analyses on the transmission data and transmission related data and output the results to the provider portal. The monitoring interval tracker 222 may receive an indication that a patient is associated with a particular patient care modality (e.g., during an enrollment process) and/or may store the association of the patient with the patient care modality in the one or more database(s) 110. The monitoring interval tracker 222 may determine and store an association between one or more interval extension rules and a particular patient and/or a particular clinic in the one or more database(s) 110. Operations of the monitoring interval tracker 222 are discussed in greater detail below.

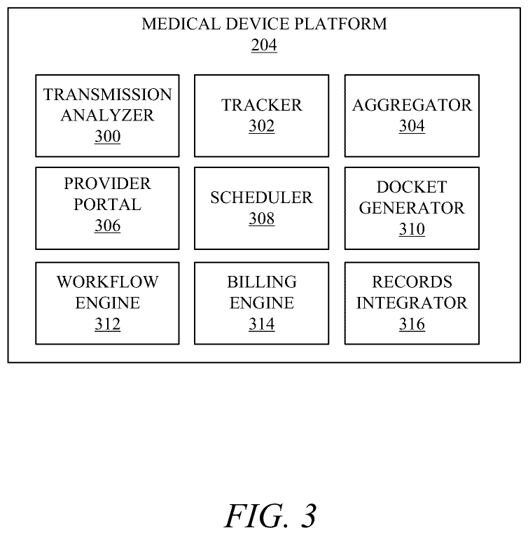

FIG. 3 is a block diagram of an example of the medical device platform 204 of FIG. 2. As depicted in FIG. 3, the medical device platform 204 may include one or more of a transmission analyzer 300, tracker 302, an aggregator 304, a provider portal 306, a scheduler 308, a docket generator 310, a workflow engine 312, a billing engine 314, and/or a records integrator 316. Other software components, data structures, applications, or modules not specifically described herein may also be included in the medical device platform 204 in other examples. Further, each of these software components may be incorporated within the monitoring interval tracker 222, the integration manager 210 and/or the cloud computing system 208, as depicted in FIG. 2.

In some examples, the transmission analyzer 300 may be configured to analyze information and data received from one or more cardiac monitoring devices and provide automated snapshots of trends of the analyzed information. In some implementations, such information may be analyzed for a particular clinic or for multiple clinics. As described above, the one or more cardiac monitoring devices may transmit stored or obtained information or data on the performance or operation of the implanted device. This information is transmitted or downloaded to the medical device platform 204 and analyzed by the transmission analyzer 300. The analysis of the information may provide particular snapshots of analyzed and/or aggregated information through a portal (described in more detail below). For example, the transmission analyzer 300 may provide information based on the type of device associated with the information or data, a manufacturer of the device, or a particular device model. Similarly, the tracker 302 may be included in the medical device platform 204 for tracking trends in the received data over time. Through the combination of the transmission analyzer 300, the tracker 302, and other software components, snapshots of device information may be provided, such as trends in docket reports receiving or missing approvals, monitoring intervals with extended DOSs, overall transmissions of data received, and actions needed for particular monitoring intervals. More analytics and information concerning the received data from the one or more medical devices and analyzed by the transmission analyzer 300 are discussed in more detail below.

In some examples, the aggregator 304 may also be included in the medical device platform 204. In general, the aggregator 304 may be configured to retrieve the diagnostic and other device-related data from each of the manufacturer systems 206 discussed above and/or information from the cardiac monitoring device. In an example, the aggregator 304 may utilize manufacturer-specific courier engines to retrieve the data using the particular security measures, communication protocols, data formats, and other characteristics of the specific manufacturer system 206 required to retrieve the data therefrom. In at least some examples, the use of the aggregator 304 may reduce the need for in-clinic information technology (IT) specialists to retrieve the diagnostic data and other information from the implantable medical devices, especially for devices from multiple manufacturers, each of which may employ their own data formats, communication protocols, and the like.

In some examples, the provider portal 306 may be configured to present a web interface to the clinic systems 202 that facilitates access by clinic staff to the provider-oriented device data information corresponding to the patients of that clinic. The provider portal 306 and may utilize a log on of the clinic staff and the patient, respectively, to allow access to the provider-oriented or patient-oriented information, as appropriate. In some examples, logging on to provider portal 306 may facilitate access by clinic staff to other systems external to or located within the clinic system 202 (e.g. the medical records manager 216 and/or the HIE system 220 of FIG. 2) via single sign-on (SSO) functionality, as mentioned above. In addition, the provider portal 306 may provide an application programming interface (API) that facilitates patient or provider access to electronic health records (EHRs) of the patient that may contain access points, such as, for example, embedded web links, to the device-related information. The provider portal 306 may present one or more visualizations of the monitoring interval tracker 222, such as timelines, interval shapes, indicators, blocks, and/or icons representing monitoring intervals, docket reports, approval dates, and DOSs, as discussed in greater detail below.

In some examples, the medical device platform 204 may provide or include other information portals aside from the provider portal 306, such as, portals for administrative personnel associated with a clinic or insurance company, executives associated with a clinic or insurance company, employees of one or more cardiac device manufacturers, and so on. In such examples, each particular class or group of potential users of the medical device platform 204 may be associated with a particular access scope or set of access rights, set of security requirements (e.g., requirements for user names, passwords, computer systems, etc.). As a result, each group of users may employ a corresponding user portal similar to the provider portal 306. Each particular portal may be accessible by way of different Uniform Resource Locators (URLs), or may be distinguished in one or more other ways.

In some examples, the scheduler 308 may be configured to present a web interface (e.g., the web portals described above, or a separate web portal) accessible to patients and/or clinic staff to schedule appointments, such as in-office or in-home device check or programming visits, with clinic patients. In some examples, the scheduling web portal may be customized for each particular clinic, possibly providing additional information regarding services rendered by the clinic, descriptions of the members of the clinic staff, and so on. For instance, the monitoring interval tracker 222 may generate an action needed indicator corresponding to a cardiac monitoring device having in-office care. Receiving a user input at the action needed indicator may cause the scheduler 308 to initiate a scheduling procedure for the particular patient being monitored by the cardiac monitoring device.

The docket generator 310 may be configured to create one or more dockets (e.g., “docket reports”) associated with a patient or a collection of received data. In general, the docket report is a report of sorts that summarizes or otherwise provides interpretations and records of received patient CIED transmissions. All or portions of the docket report may be populated with information received during transmissions and provided to clinic staff for analysis and approval. The docket report may include such information as device manufacturer information, clinic staff approval, clinical data, care plans, audit trails for the device, patient information, summary statements of data analysis, and the like. Any information concerning the patient information or information received from multiple medical devices may be included in the docket through the docket generator 310. In some instances, the monitoring interval tracker 222 may, in response to receiving transmission data, generate a docket report needed indicator which, upon receiving a user input, causes the docket generator 310 to initiate a docket creating process for the received transmission.

The workflow engine 312 may be configured to monitor information regarding periodic device checks, the resulting diagnostic data, and other information related to the cardiac monitoring devices of one or more patients, and based on that information, recommend changes to the workflow of the clinic, such as, for example, changes to device check schedules, changes to the particular types of information retrieved from the implantable medical device, changes to how the retrieved information is processed, and the like, thus possibly rendering the operations of the clinic more efficient.

The billing engine 314 may be configured to present an interface (e.g., via the provider-oriented web portal) through which clinical staff may enter an indication of one or more clinical actions taken with respect to an implantable medical device of a patient, such as the cardiac monitoring device, and in which a currently appropriate billing code representing that action is generated. Further, the resulting billing codes for one or more such actions may further be inserted into a billing code summary sheet or other format for presentation to the patient, medical insurance company, and so on. In some examples, the billing engine 314 may receive or retrieve information regarding changes in billing codes from the Centers for Medicare and Medicaid Services (CMS) employable in a prospective payment system (PPS) and utilize those changes to update the billing codes corresponding to clinical actions related to implantable medical devices.

The records integrator 316 may be configured to update or populate EHRs with processed diagnostic data and other information related to implantable medical devices by, for example, embedding web links into the EHR of a patient that facilitate access to the processed device-related data. Such data may include, for example, dates of the diagnostics performed on the implantable medical device, numerical data and/or graphs of the diagnostics results, recommended and/or performed actions based on the diagnostic results, the health or biological response of the patient to the operation of the device based on data detected by the device, and so on.

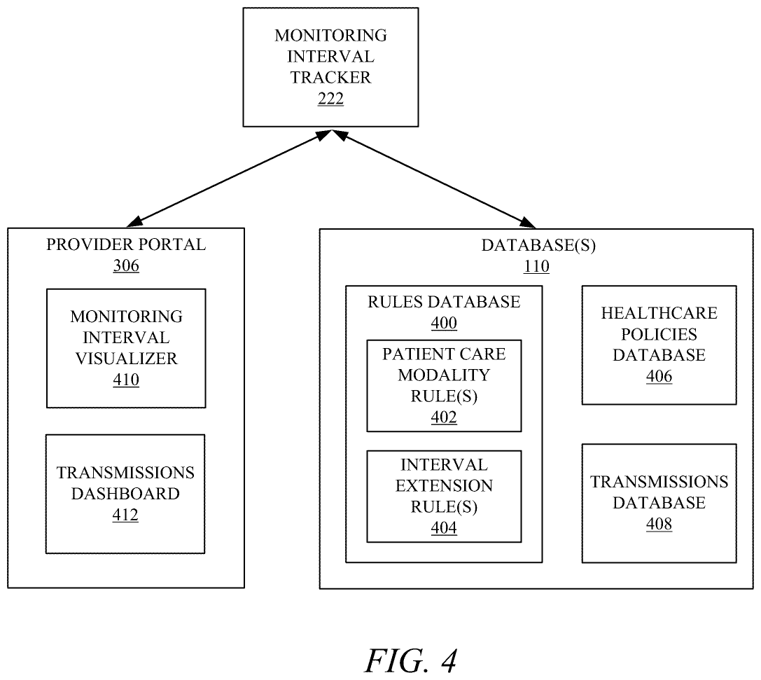

FIG. 4 is a block diagram of an example of the monitoring interval tracker 222 of FIG. 2. The monitoring interval tracker 222 may include many of the software components discussed above regarding FIG. 3 to perform operations such as analyzing received transmission data to identify the cardiac monitoring device from which the transmission data originates, categorize the transmission data according to one or more patient care modalities being provided for the cardiac monitoring device, generate one or more dockets for the transmission data, extend one or more monitoring intervals for the cardiac monitoring device, and/or generate a DOS for the transmission data. The monitoring interval tracker 222 may access information stored in the database(s) 110. For instance, the database(s) 110 may include a rules database 400 for storing one or more patient care modality rule(s) 402 and/or one or more interval extension rule(s) 404. The patient care modality rule(s) 402 may include a first patient care modality rule for heart failure patient care indicating that if a monitoring interval is associated with heart failure patient care, the monitoring interval has a 31-day length of time. The patient care modality rule(s) 402 may include a second patient care modality rule for remote monitoring patient care indicating that if the monitoring interval is associated with remote monitoring patient care, the monitoring interval has a 91-day length of time. The patient care modality rule(s) 402 may include a third patient care modality rule for in-office patient care indicating that if the monitoring interval is associated with in-office patient care, the monitoring interval has a 91-day length of time. The patient care modality rule(s) 402 may include additional patient care modality rules for other types of patient care indicating that the monitoring interval has other lengths of time. According to the patient care modality rule(s), an end date of the monitoring interval may be a predicted DOS for transmission data received during the monitoring interval (i.e., prior to the end date) should all DOS criteria be met during the monitoring interval. However, in some cases, the DOS criteria may not be met before the end date, in which case the monitoring interval tracker 222 may access the interval extension rule(s) to generate an extended end date for the monitoring interval.

In some examples, the interval extension rule(s) 404 may indicate how the monitoring interval is to be extended, not extended, or closed, based on the transmission data and transmission related data. For instance, the interval extension rule(s) 404 may include a first interval extension rule (e.g., a “transmission roll-by-day” rule) indicating that the DOS is the end date of the monitoring period or a received date of the transmission data—whichever is later. According to the transmission roll-by-day rule, should the end date of the monitoring interval occur and the transmission data is yet to be received, the monitoring interval tracker 222 is to generate an extended end date, adding one day to the monitoring interval each day until the transmission data is received. Once the transmission data is received, the monitoring interval will end, the DOS will be generated for the monitoring interval as the received date of the transmission (which is also a final extended end date of the monitoring interval), and a second monitoring interval will be generated to run subsequent to the ended or closed monitoring interval. According to the transmission roll-by-day rule the transmission data may still require a docket report and an approval for the docket report for billing purposes, but the second monitoring interval will still start on the date after the received date of the transmission so as not to be dependent on physician or medical personnel timeliness for generating and approving the docket report of the transmission data.

In some examples the interval extension rule(s) 404 may include a second interval extension rule (e.g., a “roll-by-interval” rule) indicating that the DOS is the end date of the monitoring period regardless of whether transmission data is received during the monitoring period. according to the roll-by-interval rule, should the end date of the monitoring period occur and transmission data is yet to be received, the monitoring interval will end without any extensions and may be categorized as a “missed” monitoring interval for patient follow-up and billing procedures. A second monitoring interval will be generated to run subsequent to the ended or closed monitoring interval. The second monitoring interval may have a length of time that is the same as a length of time for the closed monitoring period. No docket report or approval requirement will be generated for the closed monitoring period.

In some examples, the interval extension rule(s) 404 may include a third interval extension rule (e.g., an “approval roll-by-by day” rule) indicating that the DOS is the end date of the monitoring period or an approval date (e.g., indicated by an approval timestamp) for a docket report corresponding to the transmission data-whichever is later. According to the approval roll-by-day rule, should the end date of the monitoring interval occur and the approval timestamp for the docket report is yet to be received, the monitoring interval tracker 222 is to generate the extended end date, adding one day to the monitoring interval each day until the approval timestamp is received. Once the approval timestamp is received, the monitoring interval will end, the DOS will be garneted for the monitoring interval as the approval date for the docket report (which is also a final extended end date of the monitoring interval), and a second monitoring interval will be generated to run subsequent to the ended or closed monitoring interval. In some instances, the medical device platform 204 may generate multiple docket reports during the monitoring interval, in which case the earliest approval timestamp corresponding to one of the docket reports of the multiple docket reports will be determined to be the DOS date for the monitoring interval.

In some examples, the patient care modality rule(s) 402 and/or the interval extension rule(s) 404 may be based on one or more healthcare polices, for instance, stored in a healthcare policies database 406. The healthcare policies database 406 may store one or more Heart Rhythm Society (HRS) care guidelines and/or Center for Medicare and Medicaid Services (CMS) care guidelines. For instance, the CMS care guidelines may include one or more professional codes (PC), such as PC 93294 stating that “Interrogation device evaluation(s) (remote), up to 90 days; single, dual, or multiple lead pacemaker system with interim analysis, review(s) and report(s) by a physician or other qualified health care professional (Do not report 93294 in conjunction with 93288, 93293) (Report 93294 only once per 90 days);” PC 93295 stating that “Interrogation device evaluation(s) (remote), up to 90 days; single, dual, or multiple lead implantable defibrillator system with interim analysis, review(s) and report(s) by a physician or other qualified health care professional (For remote monitoring of physiologic cardiovascular data elements derived from an ICD, use 93297) (Do not report 93295 in conjunction with 93289) (Report 93295 only once per 90 days);” PC 93297 stating that “Interrogation device evaluation(s), (remote) up to 30 days; implantable cardiovascular monitor system, including analysis of 1 or more recorded physiologic cardiovascular data elements from all internal and external sensors, analysis, review(s) and report(s) by a physician or other qualified health care professional (For heart rhythm derived data elements, use 93295) (Do not report 93297 in conjunction with 93290, 93298) (Report 93297 only once per 30 days);” and/or PC 93298 stating that “Interrogation device evaluation(s), (remote) up to 30 days; implantable loop recorder system, including analysis of recorded heart rhythm data, analysis, review(s) and report(s) by a physician or other qualified health care professional (Do not report 93298 in conjunction with 33282, 93291, 93297) (Report 93298 only once per 30 days).” The monitoring interval tracker 222 (and/or other components of the medical device platform 204 may access the healthcare policies, such as PC codes, and generate the patient care modality rule(s) and/or the interval extension rule(s) to correspond to PC codes (e.g., via manual entries and/or automated text and content recognition with machine-learning techniques). The healthcare polices database may be updated at regular intervals (e.g., weekly, monthly, annually, etc.) to reflect any HRS or CMS policy changes, such that the patient care modality rule(s) and/or interval extension rule(s) 404 are continually up-to-date to provide accurate monitoring interval tracking that maps to a highly efficient billing technique.

In some examples, the database(s) 110 may include a transmissions database to store the transmission data and transmission related data associated with the transmission data. For instance, the transmission related data associated with the transmission may include a received date timestamp indicating the received date of the transmission data, a cardiac monitoring device identifier corresponding to the cardiac monitoring device from which the transmission data originated, a patient identifier, a clinic identifier, a docket report creation date timestamp, an approval timestamp, an indication of DOS criteria status, and/or any data generated or received by other components of the medical device platform 204 (e.g., the integration manager 210, the cloud computing system 208, the transmission analyzer 300, the tracker 302, the aggregator 304, etc.) related to the transmission data.

In some examples, the monitoring interval tracker 222 may generate a monitoring interval visualizer 410 for presenting the monitoring intervals and indications of the end date, the extended end date, a docket creation date, the approval date, and/or the DOS. The monitoring interval visualizer 410 may present “action needed” indications to alert medical personal that a particular monitoring interval needs a transmission, needs a docket report, or needs approval for a docket report. The indications may be presented as selectable shapes and/or interactive graphical user interface (GUI) indicators via the provider portal for providing additional information upon receiving user input. The monitoring interval visualizer 410 is discussed in greater detail below.

In some examples, the monitoring interval tracker 222 may generate may generate a transmissions dashboard 412. For instance, the monitoring interval tracker 222 may access the transmission data and/or transmission related data from the transmissions database 408, aggregate the transmission data and/or transmission related data, perform analysis on the aggregated transmission data and/or transmission related data, and present results of the analysis at the transmissions dashboard 412. In some examples, the transmission data and/or transmission related data may be aggregated according to a particular clinic identifier so that the results are transmission metrics for a particular clinic corresponding to the particular clinic identifier. The transmissions dashboard 412 may be presented via the provider portal 306. The transmissions dashboard 412 is discussed in greater detail below.

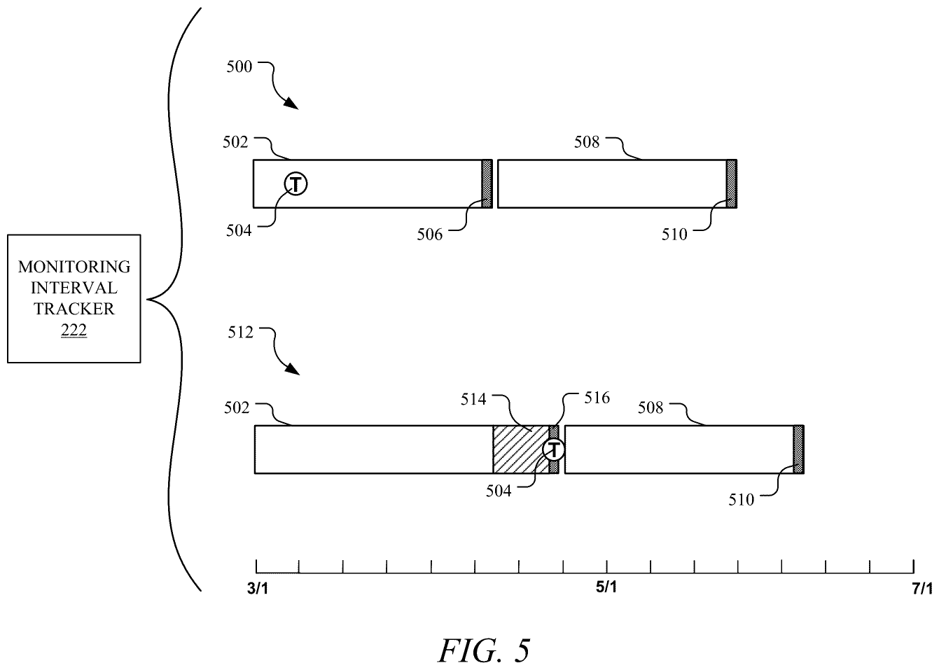

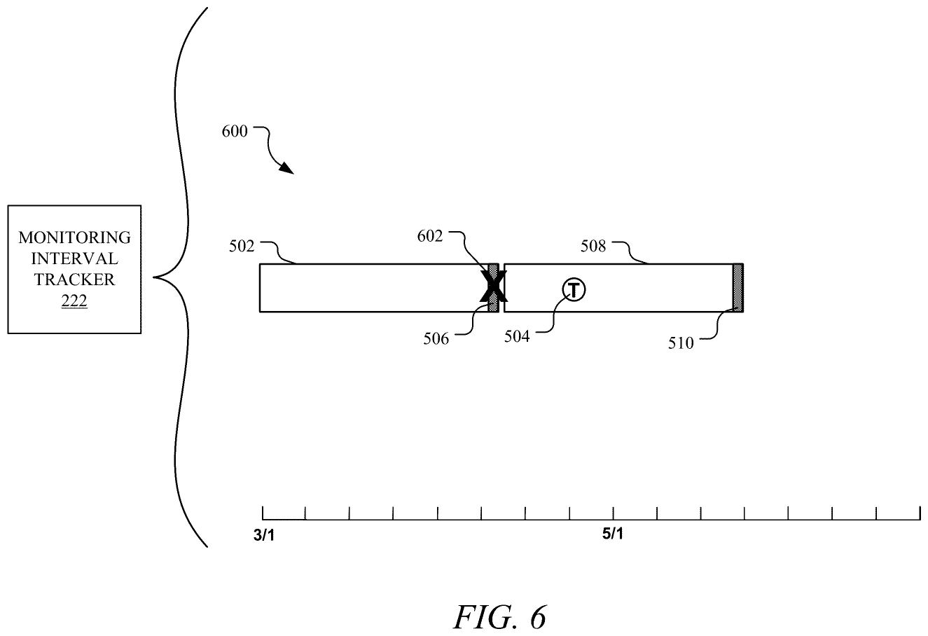

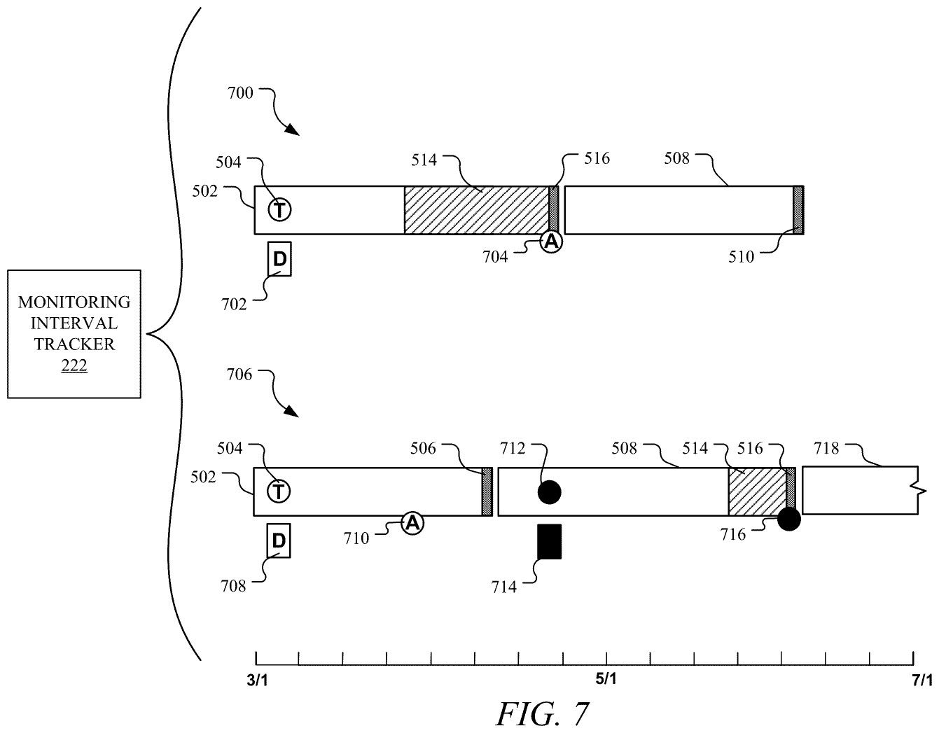

FIG. 5 illustrate multiple example timeline diagrams of operations performed by the monitoring interval tracker 222 using the transmission roll-by-day rule to generate one or more DOSs. FIG. 6 illustrates an example timeline diagram of operations performed by the monitoring interval tracker 222 using the transmission roll-by-interval rule to generate one or more DOSs. FIGS. 7-11 illustrate multiple example timeline diagrams of operations performed by the monitoring interval tracker 222 using the approval roll-by-day rule to generate one or more DOSs.

Turning to FIG. 5, two example timeline diagrams are illustrated showing operations performed by the monitoring interval tracker 222 using the transmission roll by-day rule to generate one or more DOSs, generate a new monitoring interval, and/or generate an extension to a monitoring interval. For instance, in a first example scenario 500, a first monitoring interval 502 may be generated. A trigger may occur when transmission data 504 occurs, which is defined as a “success” when the transmission data 504 is received during the first monitoring interval 502 (i.e., prior to an end date 506 of the first monitoring interval 502). In response to a “success,” the monitoring interval tracker generates the DOS as being the end date 506 of the first monitoring interval 502 and generates a new or second monitoring interval 508 to run subsequent to the first monitoring interval. The second monitoring interval 508 has a second end date 510 defining a length of time that is the same as the length of time for the first monitoring interval 502 (which may be based on the patient care modality rule(s) 404). A second example scenario 512 is illustrated where the transmission data 504 is not received during the first monitoring interval 502 (i.e., prior to the end date 506), which is defined as a “failure” according to the transmission roll-by-day rule. Therefore, the monitoring interval tracker 222 may generate an interval extension 514, extending the interval out by a day until the transmission data 504 is received, for instance, on an extended end date 516. Upon receiving the transmission data 504 during the interval extension 514 and on the extended end date 516, the monitoring interval tracker generates the DOS (e.g., a finalized DOS in contrast to the initial, predicted DOS prior to generating the interval extension) as being the received date of the transmission data 504 (i.e., the extended end date 516) and generates the new or the second monitoring interval 508 to run subsequent to the first monitoring interval 502. The second monitoring interval 508 has the second end date 510 defining a length of time that is the same as the length of time for the first monitoring interval 502 prior to adding the interval extension 514.

FIG. 6 illustrates an example timeline diagram showing operations performed by the monitoring interval tracker 222 using the transmission roll-by-interval rule to generate one or more DOSs and generate a new monitoring interval. In an example scenario 600, the transmission data 504 is not received during the first monitoring interval 502 (i.e., prior to the end date 506), which the transmission roll-by-interval rule defines as a “failure.” In response to the failure, the monitoring interval tracker 222 closes the first monitoring interval 502 such that it becomes a closed or missed monitoring interval 602, and the monitoring interval generates the new or second monitoring interval 508 to run subsequent to the missed monitoring interval 602. The monitoring interval tracker 222 does not generate a DOS for the missed monitoring interval. The second monitoring interval 508 has the second end date 510 defining a length of time that is the same as the length of time for the missed monitoring interval 602.

FIG. 7 illustrates two example timeline diagrams showing operations performed by the monitoring interval tracker 222 using the approval roll-by-day rule to generate one or more DOSs, interval extensions, and/or new monitoring intervals. For instance, in a first example scenario 700, the monitoring interval tracker 222 may generate the first monitoring interval 502. A “success” is defined when a docket report 702 and an approval date 704 for the docket report 702 are generated during the first monitoring interval 502 (i.e., before the end date 506 of the first monitoring interval 502). In response to the success, the monitoring interval tracker 222 generates the DOS as the end date 506 and generates the second monitoring interval 508. However, if the approval date does not occur before the date 506 (as shown in the first example scenario 700), a “failure” occurs and the monitoring interval tracker 222 generates the interval extension 514 to extend the first monitoring interval 502 by a day until the approval date 704 occurs. The monitoring interval tracker 222 generates the DOS for the first monitoring interval as the approval date 704 (i.e., the extended end date 516) and generates the second monitoring interval 508 to run subsequent to the first monitoring interval 502. The second monitoring interval 508 has the second end date 510 defining a length of time that is the same as the length of time for the first monitoring interval 502 prior to adding the interval extension 514. In a second example scenario 706, the first monitoring interval 502 is defined by the approval roll-by-day rule as “success” because a first docket report 708 is created prior to the end date 506 and a first approval date 710 for the first docket report occurs prior to the end date 506. As such, the DOS criteria for the first monitoring interval 502 is satisfied and a first DOS is generated as the end date 506, and the second monitoring interval 508 is generated. Second transmission data 712 is received during the second monitoring interval 508 (i.e., before the second end date 510) and a second docket report 714 is generated for the second transmission data 712 and during the second monitoring interval 508 (i.e., before the second end date 510). However, no approval date for the second monitoring interval 508 is received prior to the second end date 510, so the monitoring interval tracker 222 generates the interval extension 514 for the second monitoring interval 508 until a second approval date 716 occurs, satisfying the DOS criteria for the second monitoring interval 508. Accordingly, the monitoring interval tracker 222 generates a second DOS as the second approval date 716 (i.e., the extended end date 516) for the second monitoring interval 508, and generates a third monitoring interval 718, which may have a length of time that is the same length of time as the second monitoring interval 508 prior to the interval extension 514.

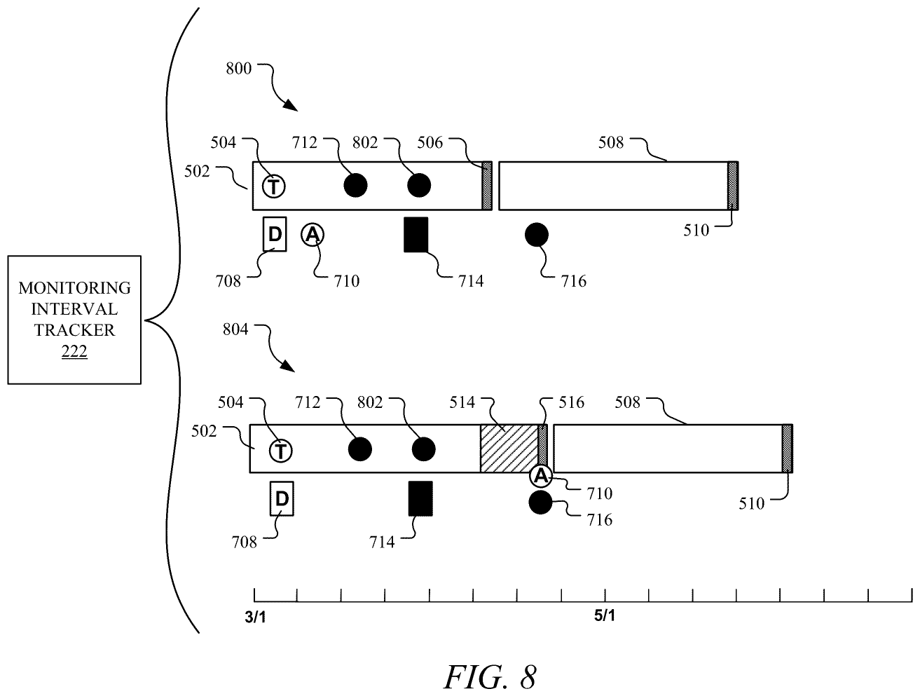

FIG. 8 illustrates two example timeline diagrams showing operations performed by the monitoring interval tracker 222 using the approval roll-by-day rule to generate one or more DOSs, interval extensions, and/or new monitoring intervals. For instance, in a first example scenario 800, the monitoring interval tracker 222 may generate the first monitoring interval 502. A “success” occurs because the first transmission data 504, the first docket report 708 for the first transmission, and the first approval date 710 for the first docket report 708 are generated during the first monitoring interval 502 (i.e., before the end date 506 of the first monitoring interval 502). The second transmission data 712, a third transmission data 802, and the second docket report 714 (e.g., for the second transmission data 712 or the third transmission data 802) may also occur during the first monitoring interval 502 before the end date 506. The second docket report 714 may lack the second approval date 716 within the first monitoring interval (i.e., prior to the end date 506). However, because the DOS criteria is met at least for the first transmission data 504, the DOS is generated for the first monitoring interval 502 at the end date 506 without an interval extension, regardless of the second approval date 716 not occurring within the first monitoring interval 502. The second approval date 716 may occur in the second monitoring interval 508, yet the second approval date 716 will not trigger the DOS for the second monitoring interval 508 because the second approval date 716 is for the second docket report 714 in the previous, first monitoring interval 502—just the second approval date 716 without corresponding transmission data or a docket report in the second monitoring interval 508 does not satisfy the DOS criteria for the second monitoring interval 508. In a second example scenario 804, the first transmission data 504, the second transmission data 712, and the third transmission data 802 may be received during the first monitoring interval 502. The first docket report 708 may be generated for the first transmission data 504 and the second docket report 714 may be generated for the third transmission data 802. Upon an occurrence of the end date 506, an approval for the first docket report 708 and the second docket report 714 is yet to be received, so the monitoring interval tracker 222 generates the interval extension 514 by day until the first approval date 710 is received and the DOS is generated as the first approval date 710 (i.e., the extended end date 506). The second approval date 716 may also be received on the extended end date 516 as well, however, the second approval date 716 has no impact on the DOS calculation for the first monitoring interval 504 because the first approval date 710 already satisfied the DOS criteria for the first monitoring interval 504.

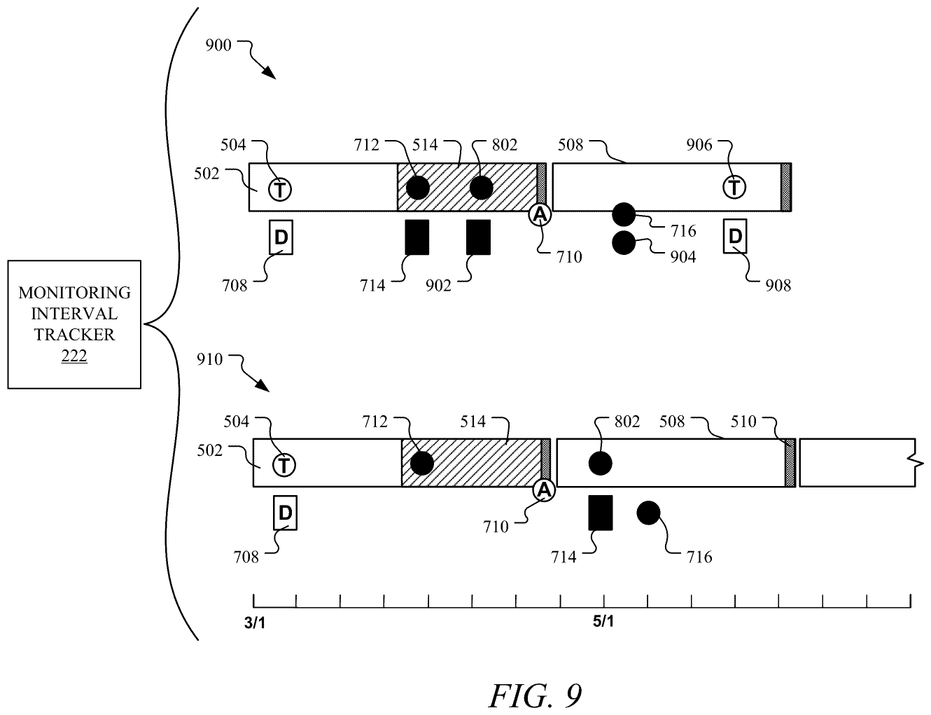

FIG. 9 illustrates two example timeline diagrams showing operations performed by the monitoring interval tracker 222 using the approval roll-by-day rule to generate one or more DOSs, interval extensions, and/or new monitoring intervals. For instance, in a first example scenario 900, the first transmission data 504 and the first docket report 708 for the first transmission data 504 may be received during the first monitoring interval 502. The monitoring interval tracker 222 may generate the interval extension 514 based on a lack of the first approval date 710 prior to the first end date 506. The second transmission data 712, and the third transmission data 802 may be received during the interval extension 514 of first monitoring interval 502. The second docket report 714 may be generated for the second transmission data 712 and a third docket report 902 may be generated for third transmission data 802 during the interval extension 514. The interval extension 514 may extend the first monitoring interval by day until the first approval date 710 is received and the DOS is generated as the first approval date 710 (i.e., the extended end date 506). The second approval date 716 for the second docket report 714 and a third approval date 904 for the third docket report 902 may occur during the second monitoring interval 508. Moreover, a fourth transmission data 906 and a fourth docket report 908 may occur during the second monitoring interval 508. However, DOS criteria for the second monitoring interval 508 is still not satisfied because the second approval date 716 and the third approval date 904 do not correspond to the fourth transmission data 906 or the fourth docket report 908 (which occurred during the second monitoring interval 508), but rather to the second docket report 714 and the third docket report 902 (which occurred during the first monitoring interval 502 and/or during the interval extension 514 of the first monitoring interval). According to the approval roll-by-day rule, approval dates for dockets and/or transmission dates occurring in previous monitoring intervals do not satisfy DOS criteria for the current monitoring interval. In a second example scenario 910 the first transmission data 504 and the first docket report 708 for the first transmission data 504 may be received during the first monitoring interval 504. The monitoring interval tracker 222 may generate the interval extension 514 based on a lack of the first approval date 710 prior to the first end date 506. The second transmission data 712 may occur during the interval extensions 514. The first approval date 710 may occur at the extended end date 516 which may satisfy the DOS criteria for the first monitoring interval 502 causing the monitoring interval tracker to generate the DOS for the first monitoring interval 502 and generating the second monitoring interval 508. The second transmission data 712 may not receive a reporting docket or approval, yet this will have no effect on the DOS for the first monitoring interval because the DOS criteria is already satisfied with the first transmission data 504, the first docket report 708, and the first approval 704. Third transmission data 802 may be received during the second monitoring interval 508, and the second docket report 714 may be generated during the second monitoring interval 508 and may correspond to the third transmission data 802 of the second monitoring interval 508 as well as the second transmission data 712 of the first monitoring interval (which did not receive a docket report during the first monitoring interval 502). The second approval date 716 for the second docket report 714 may occur during the second monitoring interval 508 satisfying the DOS criteria for the second monitoring interval and causing the DOS to be generated as the end date 510.

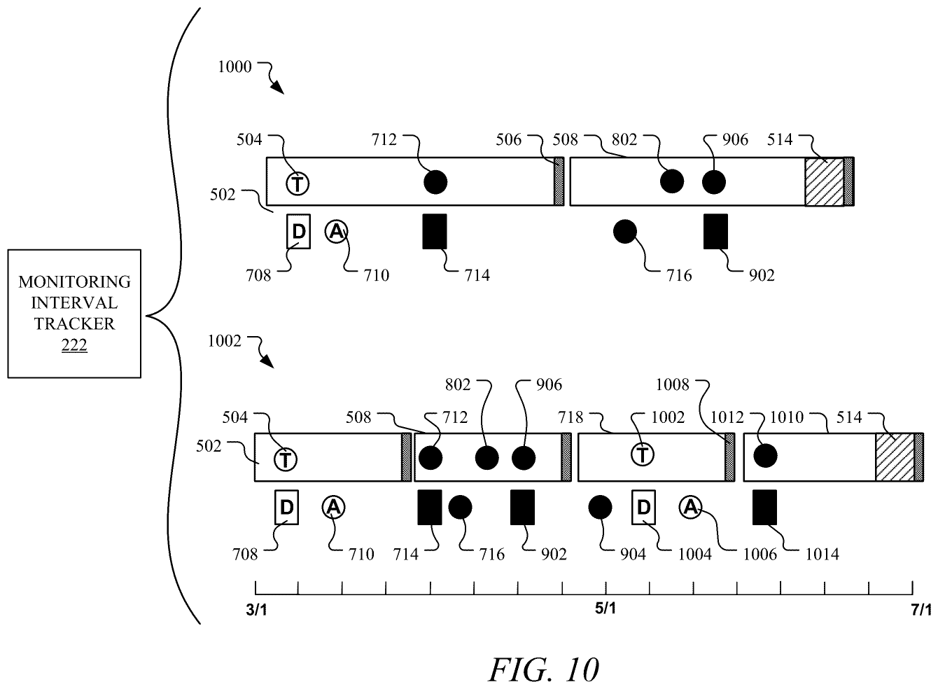

FIG. 10 illustrates two example timeline diagrams showing operations performed by the monitoring interval tracker 222 using the approval roll-by-day rule to generate one or more DOSs, interval extensions, and/or new monitoring intervals. For instance, in a first example scenario 1000, the first transmission data 504, the first docket report 708 for the first transmission data 504, and the first approval date 710 may be received during the first monitoring interval 502. the second transmission data 712 and the second docket report 714 may also be received during the first monitoring interval 502, but without receiving the second approval date 716 within the first monitoring interval. However, because the first transmission data 504, the first docket report 708, and the first approval date 710 satisfy the DOS criteria, the first monitoring interval 502 does not extend and the DOS is generated as the first end date 506. The second transmission data 712 and the second docket report 714 do not impact the first monitoring interval. The second approval 716 for the second docket report 714 may be received during the second monitoring interval 508. Additionally, the third transmission data 802, the fourth transmission data 906, and the third docket report 902 for the third transmission data 802 and/or the fourth transmission data 906 may be received during the second monitoring interval 508. However, upon occurrence of the second end date 510, the monitoring interval tracker 222 will generate the interval extension 514 for the second monitoring interval 508 by day until an approval date is received because the second approval date 716 (which occurred during the second monitoring interval 508) does not correspond to a transmission data or a docket report that also occurred in the second monitoring interval 508 (e.g., the third transmission data 802, the fourth transmission data 906, or the third docket report 902). Rather, the second approval date 716 corresponds to the second docket report 714 from the previous, first monitoring interval 502. DOS criteria is not satisfied for the second monitoring interval 508. In a second example scenario 1002 the first transmission data 504 and the first docket report 708 for the first transmission data 504 may be received during the first monitoring interval 502. The DOS criteria is satisfied and the monitoring interval tracker 222 generates the DOS as being the first end date 506 and generates the second monitoring interval 508 to run subsequent to the first monitoring interval 502. The second monitoring interval 508 has the second end date 510 defining a length of time that is the same as the length of time for the first monitoring interval 502. The second transmission data 712, the third transmission data 802, and the fourth transmission data 906 are received during the second monitoring interval 508. The second docket report 714 for the second transmission data 712 and the second approval date 716 for the second docket report 714 are also received during the second monitoring interval 508, satisfying the DOS criteria for the second monitoring interval 508. The third docket report 902 may be received for the third transmission data 802 or the fourth transmission data 906, and yet the third approval date 904 for the third docket may not be received before the second end date 510 of the second monitoring interval 508. Nevertheless, the monitoring interval tracker 222 may generate the DOS as the second end date 510 based on the DOS criteria previously being satisfied by the second approval date 716, the second docket report 714, and the second transmission data 712. The third approval date 904 may be received during the third monitoring interval 718, yet the third approval date 904 will not be considered DOS criteria for the third monitoring interval 718 because it corresponds to a docket report from a previous monitoring interval (e.g., the third docket report 902). Nevertheless, fifth transmission data 1002, a fourth docket report 1004 for the fifth transmission data 1002, and a fourth approval date 1006 for the fourth docket report 1004 may still be generated during the third monitoring interval 718, such that the DOS criteria is satisfied for the third monitoring interval 718, no interval extensions are needed, and the DOS for the third monitoring interval 718 is generated as a third end date 1008 of the third monitoring interval. Upon generating the DOS for the third monitoring interval 718, the monitoring interval tracker 222 may also generate the fourth monitoring interval 1010. The fourth monitoring interval may receive sixth transmission data 1012 and a fifth docket report 1014 for the sixth transmission data 1012. a fourth end date of the fourth monitoring interval 1010 may occur without receiving an approval date corresponding to the fifth docket report 1014, and so the monitoring interval tracker 222, according to the approval roll-by-day rule, may generate the interval extension 514 by day for the fourth monitoring interval 1010 until an approval date is received for the fifth docket report 1014 and the DOS criteria is satisfied for the fourth monitoring interval 1010.

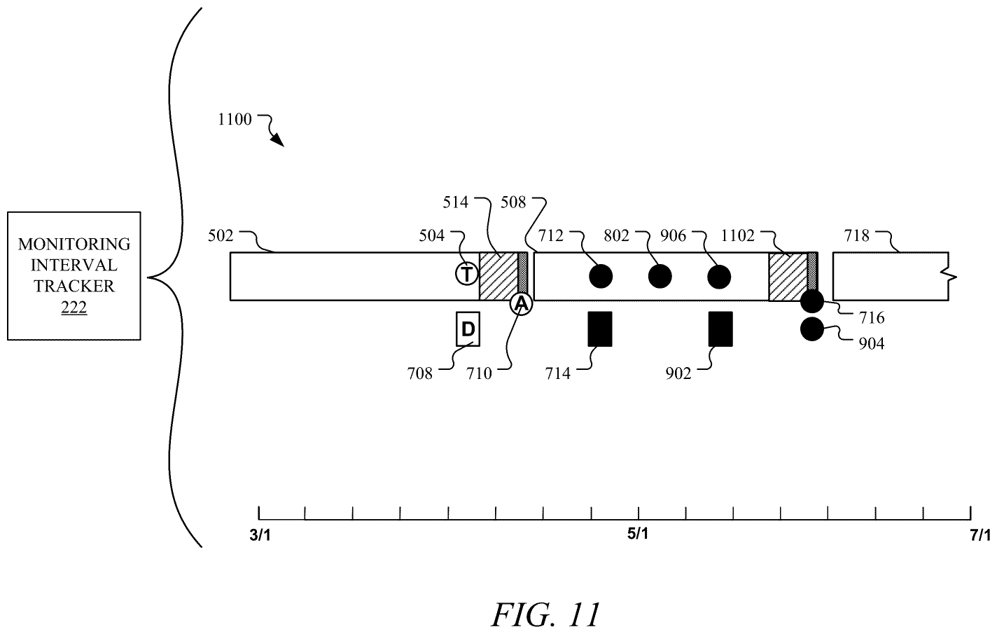

FIG. 11 illustrates an example timeline diagram showing operations performed by the monitoring interval tracker 222 using the approval roll-by-day rule to generate one or more DOSs, interval extensions, and/or new monitoring intervals. For instance, in an example scenario 1100, the first transmission data 504 and the first docket report 708 for the first transmission data 504 are received during the first monitoring interval. However, the DOS criteria is not satisfied upon occurrence of the first end date 506, so the monitoring interval tracker 222 generates the interval extension 514 to the first monitoring interval 502 until the first approval date 710 is received. The DOS is generated for the first monitoring interval 502 as the first approval date (i.e., the extended end date 516, and the second monitoring interval 508 is generated, and the first approval date 710 may be received during the first monitoring interval 502. During the second monitoring interval 508, the monitoring interval tracker 222 may receive the second transmission data 712, the third transmission data 802, and the third docket report 902 for the second transmission data 712 and/or the third transmission data 802. Upon an occurrence of the second end date 510 of the second monitoring interval 508, approval dates for the second docket report 714 and third docket report 902 are yet to be received, and so the monitoring interval tracker generates a second interval extension 1102 for the second monitoring interval 508, extending the second monitoring interval by day until the second approval date is 716 is received. Upon receiving the second approval date 716, the monitoring interval tracker determines that the DOS criteria for the second monitoring interval is satisfied and generates the DOS as the second approval date 716. The third approval date 904 for the third docket report 902 may also be received on the second approval date 716, however, the third approval date and the third docket report have no impact on the DOS for the second monitoring interval 508 because the DOS criteria for the second monitoring interval 508 is already satisfied by the second approval date 716, the second docket report 714, and the second transmission data 712. In response to generating the DOS for the second monitoring interval 508, the monitoring interval tracker 22 generates the third monitoring interval 718 having a length of time determined by a patient care modality rule.

In some examples, any of the example scenarios illustrated in FIGS. 5-11 may run concurrently with any other of the example scenarios to represent different monitoring intervals of different patient care modalities simultaneously. For instance, the first example scenario 700 may represent a heart failure care modality while the second example scenario 706 may represent a remote monitoring care modality. As such, the monitoring intervals of the first example scenario 700 may have lengths of time of 31-days while the monitoring intervals of the second example scenario 706 may have lengths of time of 91-days. A single transmission data (e.g., the first transmission data 504) may be included in multiple, concurrently running monitoring intervals, such as each monitoring interval for each type of patient care modality. Once the first transmission data 504 is received, separate docketing reports and approval dates may be required for the different patient care modalities so that DOS criteria for one patient care modality (e.g., the heart failure care modality) may be satisfied and the DOS generated while DOS criteria for another patient care modality (e.g., the remote monitoring care modality) is not satisfied, even though both concurrently running monitoring intervals are tracking the same transmission data. The ability to track different patient care modalities having different lengths of time, independently from each other, yet for a single transmission data from a single cardiac monitoring device provides significant improvements in efficiency for patient care tracking, patient follow ups, and reducing missed monitoring intervals and failures to follow up. Especially when the system is scaled up to manage hundreds or thousands of different patients having hundreds or thousands of different cardiac monitoring devices, each with their own transmission rates and combinations of different patient care modalities.

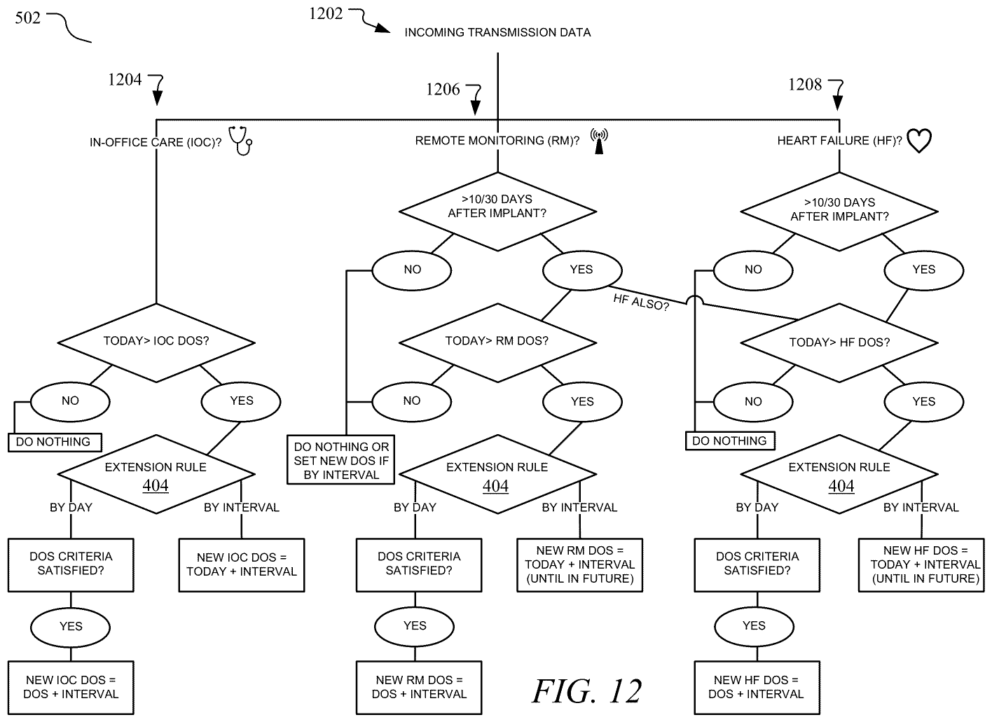

FIG. 12 illustrates an example flow chart of operations performed by the monitoring interval tracker 222 to manage cardiac monitoring devices, for instance, to generate one or more DOSs, interval extensions, and/or new monitoring intervals as discussed above. The monitoring interval tracker 222 may receive incoming transmission data 1202 representing a transmission from a cardiac monitoring device. The monitoring interval tracker 222 may receive or generate a received date timestamp indicating the received date of the transmission data 1202. The monitoring interval tracker 222 may categorize the transmission data 1202 into one or more patient care modality rules (e.g., by comparing a patient identifier, a cardiac device identifier, or other information form the incoming transmission data to information stored in the one or more database(s)), such as an in-office care modality 1204, a remote monitoring care modality 1206, and/or a heart failure care modality 1208. Should the incoming transmission data 1202 correspond to the remote monitoring care modality 1206 and/or the heart failure care modality 1208, the monitoring interval tracker 222 may calculate whether the received date is greater than 10 or 30 days after an implant date associated with the cardiac monitoring device (e.g., the association being stored in the one or more database(s) 110). If “NO”, the monitoring interval tracker 222 may determine to do nothing (i.e., omit generating a response to the transmission data), but if the incoming transmission data 1202 further corresponds to the remote monitoring care modality 1206 and a “by interval” extension rule (e.g., the transmission roll-by-interval rule), the monitoring interval tracker 222 may determine to generate a new monitoring interval with a new end date (e.g., predicted DOS). If the monitoring interval tracker calculates that the received date is greater than 10 or 30 days after an implant date, the monitoring interval tracker 222 may proceed to calculate whether “today” (i.e., the received date) is greater than the end date (e.g., the predicted DOS of the monitoring interval). Likewise, if the transmission data 1202 is categorized under the in-office care modality 1204, the monitoring interval tracker 222 may proceed directly to calculating whether “today” (i.e., the received date) is greater than the end date (e.g., the predicted DOS of the monitoring interval). If “NO,” the monitoring interval tracker 222 may determine to do nothing (i.e., omit generating a response to the transmission data), but if the incoming transmission data 1202 further corresponds to the remote monitoring care modality 1206 and a “by interval” extension rule (e.g., the transmission roll-by-interval rule), the monitoring interval tracker 222 may determine to generate a new monitoring interval with a new end date (e.g., predicted DOS). If “YES,” that is, if the monitoring interval tracker 222 calculates that “today” (i.e., the received date) is, in fact, greater than the end date (e.g., the predicted DOS of the monitoring interval), the monitoring interval tracker may, in response, access and/or use the interval extension rule 404 for the monitoring interval. If the monitoring interval tracker 222 access and/or uses the “by day” extension rule (e.g., the transmission roll-by-day rule or the approval roll-by-day rule), the monitoring interval tracker 222 may proceed to determine whether the DOS criteria for the current monitoring interval is satisfied and, if “YES,” generate a new monitoring interval with a new end date (e.g., predicted DOS) that is the previous DOS of the current monitoring interval plus a length of time based on the patient care modality rule (e.g., 1-year for the in-office care modality 1204, 91 days for the remote monitoring care modality 1206, and/or 31 days for the heart failure care modality 1208). If “NO,” that is, the monitoring interval tracker 222 determines that the DOS criteria is not satisfied while using the “by day” rule, the monitoring interval tracker 222 may continue extending the current monitoring interval by day until the DOS criteria is satisfied. If the monitoring interval tracker 222 accesses and uses the “by interval” rule, the monitoring interval may generate a new monitoring interval by calculating a new end date (e.g., predicted DOS) for the new monitoring interval that is “today” (i.e., the received date) plus the length of time based on the patient care modality rule (e.g., 1-year for the in-office care modality 1204, 91 days for the remote monitoring care modality 1206, and/or 31 days for the heart failure care modality 1208). If the monitoring interval tracker 222 determines that the DOS criteria is satisfied, the monitoring interval tracker 222 may further generate the DOS for the current monitoring that is “today” (i.e., the received date). In some examples, once a first docket is approved for a monitoring interval, DOSs may be generated for all dockets in the monitoring interval, including any subsequent dockets in the same monitoring interval.

In some instances, calculations performed by the monitoring interval tracker 222 may determine one or more states for the monitoring interval and/or may associate the state with the monitoring interval (e.g., to be reported and/or visualized via the monitoring interval visualizer 410 and/or the transmissions dashboard 412, as discussed in greater below). For instance, a monitoring interval for the remote monitoring care modality 1206 may be associated with one or more states including needs transmission (if no transmission data received during the monitoring interval), needs docket report (if no docket report generated during the monitoring interval), needs approval (if no approval for the docket report received during the monitoring interval), or satisfied (if the DOS criteria is satisfied). A monitoring interval for the heart failure care modality 1208 may be associated with one or more states including needs transmission (if no transmission data received during the monitoring interval), needs impression (if no impression received for the transmission data) needs docket report (if no docket report generated during the monitoring interval), needs approval (if no approval for the docket report received during the monitoring interval), or satisfied (if the DOS criteria is satisfied). A monitoring interval for the in-office care modality 1204 may be associated with one or more states including needs visit (if no transmission data received from a visit during the monitoring interval), needs docket report (if no docket report generated during the monitoring interval), needs approval (if no approval for the docket report received during the monitoring interval), or satisfied (if the DOS criteria is satisfied).

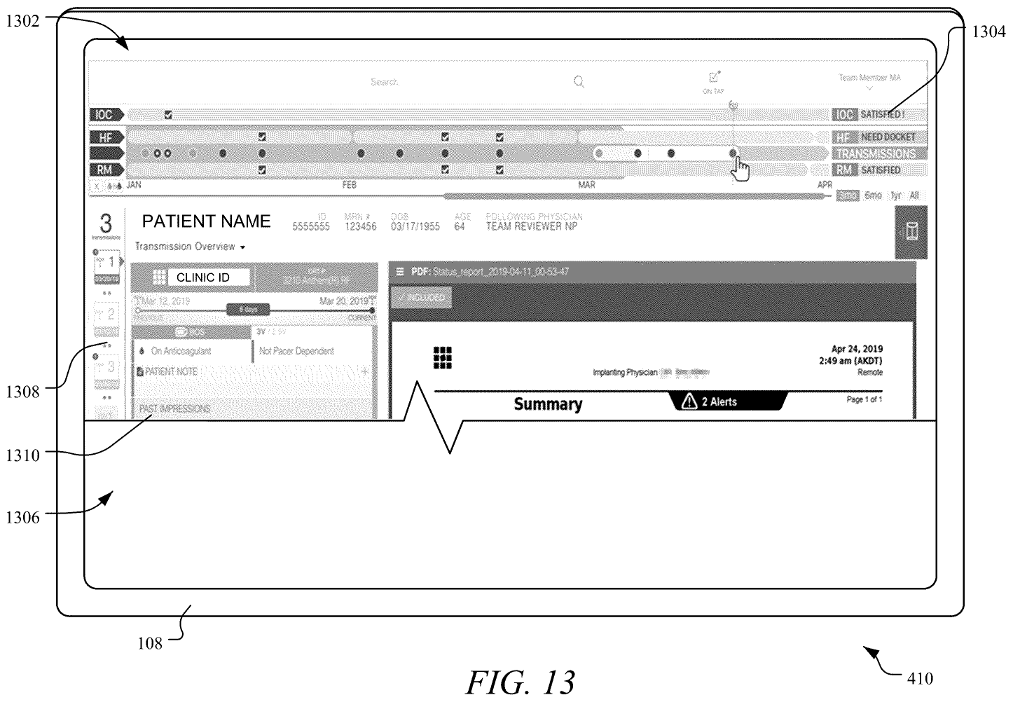

Turning now to FIGS. 13-19, various examples of a portal or user interface are provided for accessing, analyzing, observing, or otherwise managing information and data received at the medical device platform 204 from the cardiac monitoring device and/or generated by the monitoring interval tracker 222. The user interface may be displayed on a user device 108, such as that described above with relation to FIG. 1. The various user interfaces displayed on the user device 108 may be accessed through network 106 by the user device and executed by the server or servers 112 of the network environment 100. Further, one or more medical devices 104, such as one or more cardiac monitoring devices and/or implantable medical devices from one or more patients, may provide information or other operational data to the medical device manager 102 and stored in database 110. For example, utilizing a Health Level 7 (HL7) protocol, the medical device manager 102 may receive communications from device manufacturer remote transmissions that contain device and/or patient specific data and device fields for device type and model. This information may then be processed in various ways by the monitoring interval tracker 222, as discussed above, and presented or managed by the user through the user interface. Thus, a user (such as a nurse or doctor of a clinic) can receive, access, analyze, and/or manage data from the one or more medical devices 104 per patient or across multiple patients in a manner that has been previously unavailable.

As described herein, the medical device manager 102 manages CIED device transmissions and interrogations, including both in-office interrogations and remote interrogations. In one implementation, in-office interrogations utilize an in-office upload interface generated by the medical device manager 102, with which a user may drop or select one or more files for import. The files may be accessed over a network, via memory (e.g., a flash drive), and/or the like. Once the file(s) are selected, the interface may generate a visualization of a progress of the upload as the medical device manager 102 processes the interrogation data. In one implementation, an interstitial interface is generated, which may be used to verify, add, or edit encounter info, including, without limitation, an encounter date corresponding to the data extraction, a patient name, a patient date of birth, a clinic name, a device manufacturer, a device type, and a device serial number. If data is missing, the user can enter it via the interstitial interface. The medical device manager 102 may determine if the patient is a new or existing patient and proceed accordingly. Multiple visualizations are provided via the interface including a progress of processing the interrogation data, options for proceeding, editing, or cancelling, and a progress of uploading the data to the medical device manager 102. The user may further be provided with options, including whether to upload the associated PDF from the interrogation. During the processing and upload, the data file is parsed and discrete data is imported from the parsed data file. Upon import, the user can add, edit, and review the imported data via a user interface. Where the PDF is imported, it may be presented along with the imported data or otherwise be accessible via the interface. The imported data may be automatically or manually saved. Where the transmission is associated with an existing patient, a set of key identifiers are matched. The patient record shows that an in-office transmission is added to a stack of in-office interrogations, if one exists. If only a stack of remote transmissions exits or there is no current in-office interrogation stack, the medical device manager 102 generates a new docket instance. As such, the medical device manager 102 may generate and track workflows for in-office interrogations separate from remote interrogations. As such, the medical device manager 102 may generate an in-office docket and a remote docket, with the in-office docket including unique CPT and ICD-10 codes. In some examples, the medical device manager 102 generates a report (e.g., docket) documenting a trail of patient in-office interrogation review and impressions by a technician and approval of a provider. The report is generated by the medical device manager 102 for in-office or remote interrogation by combining patient information, proprietary historical presentation, a transmitted PDF file if attached by the reviewer, and/or the like. The report is generated in the cloud by the medical device manager 102 and may be stored or packaged and made available as a PDF download. Any of this information may be sent to the monitoring interval tracker 222 to perform the operations herein. Moreover, the monitoring interval tracker 222 may generate, based on outputs of the operations, the monitoring interval visualizer 410 and/or the transmissions dashboard 412, as discussed in greater detail below.

FIG. 13 shows an example monitoring interval visualizer 410. In general, the monitoring interval visualizer 410 provides data and data management options that may be a user-specific portal to the user's role (such as a nurse or doctor of a clinic or a patient analyzing their own device data) as graphical representations of the plurality of monitoring intervals. Thus, the data management options displayed in the monitoring interval visualizer may be altered or different depending upon the role of the user accessing the user interface. The medical device manager 102 may determine the style and/or content of the monitoring interval visualizer 410 based on log-in information provided to the system by the user upon accessing the system. Other interfaces discussed in more detail below may or may not be available to particular users based on similar identifying information.

In the particular example illustrated in FIG. 13, the monitoring interval visualizer 410 may include first portion 1302 for presenting plurality of timelines. For instance, a first timeline may correspond to the in-office care modality 1204, a second timeline may correspond to the remote monitoring care modality 1206, and/or a third timeline may correspond to the heart failure care modality 1208. Moreover, a fourth timeline may be presented in the first portion 1302 representing one or more received dates of the transmission data. The monitoring intervals may be presented as one or more shapes, such as blocks, on the plurality of timelines. A plurality of interactive and/or selectable indicators, such as graphical elements or icons, may be presented on the plurality of timelines to represent the received date of the transmission data and other transmission related data. For instance, a square on a timeline may represent a docket report (e.g., a creation date of the docket report), and a check mark in the square may indicate that the docket report has been approved (e.g., has been associated with an approval timestamp). The blocks may be color coded such that a first color indicates that the DOS criteria has been satisfied and/or a second color indicates that the DOS criteria has not been satisfied. In some examples, the first portion 1302 may present, at side of the plurality of timelines, one or more action needed indicator(s) 1304. The action needed indicator(s) may correspond to a patient care modality and, accordingly, be in a same row as the timeline that corresponds to the same patient care modality. The monitoring interval tracker 222 may determine, the states for current monitoring intervals of each of the timelines, such as whether the DOS criteria is met and, if the monitoring interval tracker 222 determines that a particular DOS criterion is not met, may present the action needed indicator corresponding to the unmet particular DOS criteria, indicating the state of the monitoring interval. For instance, a first action needed indicator may indicate that the docket report is needed for a particular patient care modality (e.g., the heart failure care modality). Additionally, or alternatively, the action needed indicator(s) 1304 may indicate that the transmission data is needed for a current monitoring interval, the approval of the docket report is needed for a current monitoring interval, and/or that the DOS criteria is satisfied for a current monitoring interval.

In some examples, the interactive and/or selectable indicator of the first portion 1302 may provide additional transmission related information in response to a user input or selection, such as clicking on or hovering a cursor over the interactive and/or selectable indicator. For instance, a second portion 1306 (e.g., positioned below the first portion 1302 on the display of the user device 108) may present the transmission related information corresponding to user inputs in the first portion 1302. The second portion 1306 may include, in response to a selection of a particular monitoring interval, an indication of the patient name associated with the selected monitoring interval, a patient age, a patient date of birth, a name of an assigned physician, and/or a number of transmissions that have occurred during the selected monitoring interval. The second portion 1306 may include a transmissions sidebar 1308 presenting selectable transmission indicators representing the transmissions of the selected monitoring interval and/or a date of transmission. Upon receiving a user input at the selectable transmission indicator, the second portion 1306 may present additional information corresponding to the particular transmission represented by the selectable transmission indicator, such as docket reports and past impressions for the transmission, patient notes, details of the cardiac monitoring device. The second portion 1306 may include one or more viewing window(s) 1310 for presenting the transmission related information corresponding to user inputs at the first portion 1302 and/or the transmissions sidebar 1308. In some instances, the second portion 1306 may present a completed docket report for a monitoring interval that has the DOS criteria satisfied. In some examples, upon receiving a selection of a monitoring interval that has not yet had the DOS criteria satisfied, the second portion 1306 may present an interval report summarizing transmissions, impressions, plans, and doctor notes falling within the monitoring interval. An interval report may be auto-generated for each type of patient care modality, that is, without requiring authorization. The interval report may indicate diagnostic information (e.g., indicating a “stable” status or an “elevated” status regarding heart failure diagnostics) and/or what information is awaiting approval (e.g., has a “pending” status) or has received approval (e.g., has an “approved” status) from a physician. In some instances, the monitoring interval visualizer 410 may not display a DOS (e.g., the predicted DOS) for a monitoring interval until a docket in the monitoring interval is approved. In other instances, the predicted DOS may be displayed. In some examples, the monitoring interval may not be displayed until a first docket in the monitoring interval is approved.

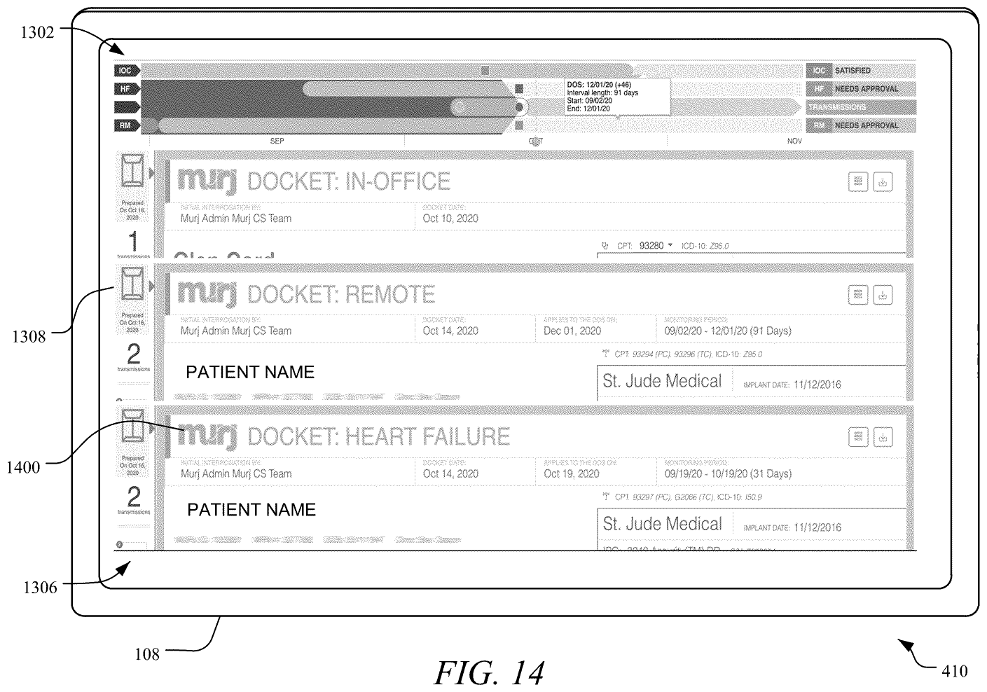

FIG. 14 illustrates an example monitoring interval visualizer 410 with a docket stacking window 1400 in the second portion 1306. For instance, upon receiving a selection in the first portion of a particular transmission data, the monitoring interval visualizer the second portion 1306 may present multiple docket reports in the docket stacking window 1400 corresponding to the selected transmission data. For instance, the docket stacking window may present a first docket corresponding to the in-office care modality 1204, a second docket report corresponding to the remote monitoring care modality 1206, and/or a third docket report corresponding to the heart failure care modality 1208. The multiple docket reports may be presented in alignment with the selectable transmission indicators of the transmissions sidebar 1308 such that the multiple docket reports appear to overlap. Upon receiving a selection of a particular docket report form the docket stacking window 1400 and/or the transmissions sidebar 1308, the monitoring interval visualizer may present an unobstructed or complete version of the selected docket report.

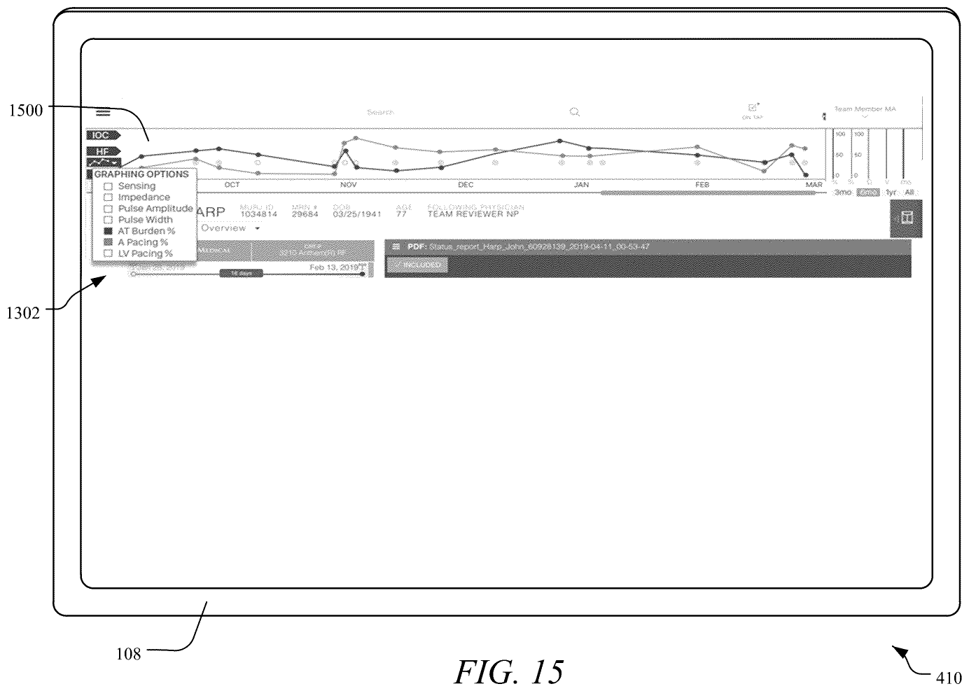

FIG. 15 illustrates an example monitoring interval visualizer 410 including a transmission details graph 1500 that may be presented in the first portion 1302. For instance, a selectable graphical indicator may, upon receiving user input, cause a list of selectable graphing options to be presented. The graphing options may include any particular value or metric that is included in or related to the transmission data (e.g., a sensing value, an impudence a pulse amplitude, a pulse width, an atrial fibrillation (AT) burden, an atrial pacing, a left ventricular pacing, etc.). Upon receiving a selection of one or more of the graphing options, the monitoring interval visualizer 410 may cause a graph (e.g., a line graph, a bar graph, etc.) to be laid over the first portion 1302 (e.g., in place of the timelines) with data points defined by each of the transmissions. The monitoring interval tracker 222 may access data stored in the transmissions database (e.g., based on timestamps and/or identifiers associated with each transmission) to generate the transmission details graph 1500.

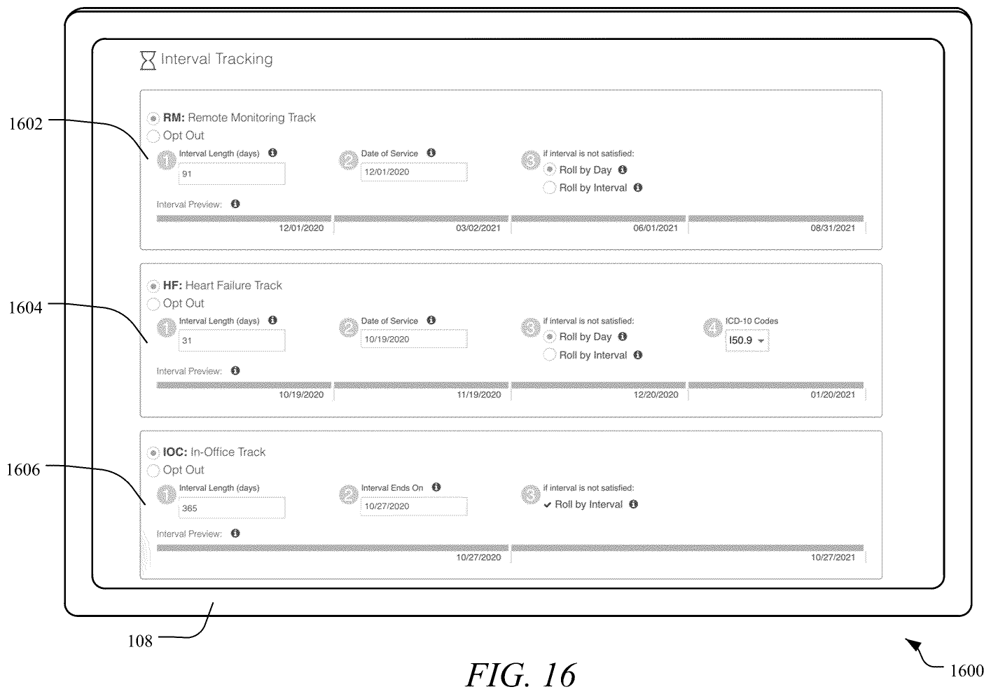

FIG. 16 illustrates an example monitoring interval configuration interface 1600 for generating parameters for the monitoring interval tracker 222. The monitoring interval configuration interface 1600 may present one or more input fields or selectable icons for generating parameters and/or indicating which of the patient care modality rule(s) 402 and/or interval extension rule(s) 404 to use for particular transmission data. The parameters generated by the monitoring interval configuration interface 1600 may, in some instances, be associated with a particular patient name or identifier or a particular cardiac monitoring device identifier and stored in the transmissions database 408. When transmission data is received, a patient name or identifier or a cardiac monitoring device identifier included in the transmission data can be matched to those stored in the transmissions database 408, and the associated parameters may be retrieved and applied to the transmission data. In some examples, the monitoring interval configuration interface 1600 may receive, at a first section 1602, information associated with the remote monitoring care modality 1206, such as an opt-in or an opt-out for this type of patient care modality, an interval length (e.g., 91 days or another interval length) an initial predicted DOS (e.g., an end date) for an initial monitoring interval, and/or whether the interval extension rule 404 is a by-interval rule or a by-day rule. The monitoring interval configuration interface 1600 may receive information, at a second section 1604, associated with the heart failure care modality 1208, such as an opt-in or an opt-out for this type of patient care modality, an interval length (e.g., 31 days or another interval length) an initial predicted DOS (e.g., an end date) for an initial monitoring interval, whether the interval extension rule 404 is a by-interval rule or a by-day rule, and/or an International Classification of Disease (ICD)-10 code associated with the patient care. The monitoring interval configuration interface 1600 may receive information, at a third section 1606, associated with the in-office care modality 1204, such as an opt-in or an opt-out for this type of patient care modality, an interval length (e.g., 365 days or another interval length) and an end date for an initial monitoring interval. In some examples, the monitoring interval configuration interface 1600 may include an interactive component for generating a new configurable patient care modality with any interval length, initial interval monitoring period, initial DOS, future DOSs, interval extension rule, and/or ICD-10 code, and adding the configurable patient care modality to the transmissions database 408 associated with the patient. The monitoring interval configuration interface 1600 may include an option for selecting a group of patients and/or classes of device types, and applying particular rules, settings, and parameters to the selected group of patients and/or classes of device types. In some examples, the monitoring interval configuration interface 1600 may present an option for changing the patient care modality rule(s) 402 and/or interval extension rule(s) 404 for a patient in response to the patient changing clinics (e.g., moving from a first clinic to a second clinic).

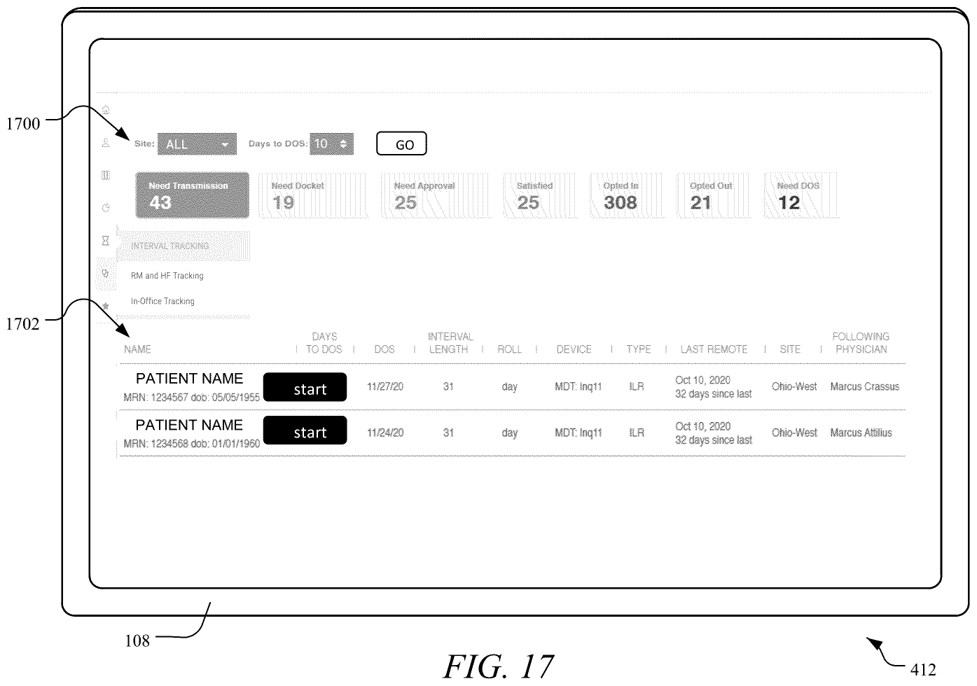

FIG. 17 illustrates an example transmissions dashboard 412 to aggregate transmission related data for a particular clinic from the transmissions database 408, generate one or more transmission metrics for the particular clinic, and present the transmission metrics on the user device 108 of the particular clinic. For instance, transmissions dashboard 412 may receive inputs at one or more interactive graphical elements indicating to which clinic sites the transmission metrics will relate, and to which patient care modality the transmission metrics will relate. The transmission metrics may be presented in a first section 1700 and may include an indication of a number of currently active monitoring intervals that need transmissions for a selected patient care modality, a number of transmissions that need a docket report for the selected patient care modality, a number of docket reports that need an approval for the selected patient care modality, a number of currently active monitoring intervals that have satisfied all DOS criteria for the selected patient care modality, a number of patients opted into the selected patient care modality, a number of patients opted out of the selected patient care modality, and a number of DOSs needed for the selected patient care modality. In some examples, the transmission metrics may comprise interactive graphical indicators such that receiving a selection or user input at the transmission metric causes additional details related to the selected transmission metric to be presented at a second section 1702 of the transmission dashboard below the first section 1700, such as a list of patients corresponding to the selected transmission metric (e.g., that need transmissions, that need a docket report, etc.), and/or additional information related to the list of patients. In some instances, the transmission metrics may be color coded with a first color indicating transmission metrics needed to satisfy a billing requirement (e.g., DOS criteria) of currently active monitoring intervals, and a second color indicating patients (e.g., listed in the second section 1702) that have satisfied the billing requirements for the currently active monitoring intervals.

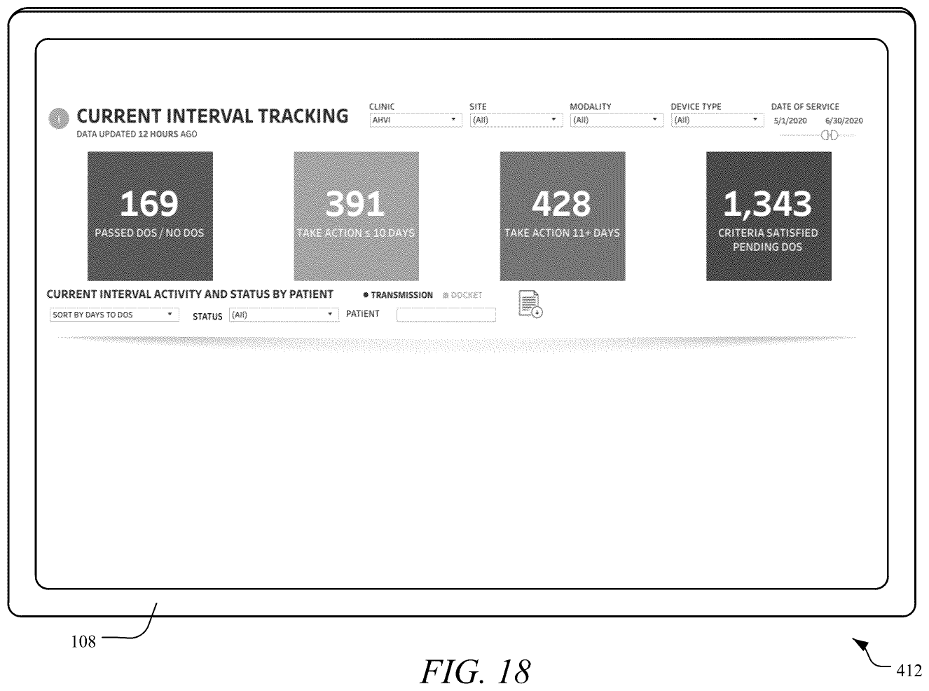

FIG. 18 illustrates an example transmissions dashboard 412 to aggregate transmission related data for a particular clinic from the transmissions database 408, generate one or more transmission metrics for the particular clinic, and present the transmission metrics on the user device 108 of the particular clinic. The transmissions dashboard 412 may help the clinic see and understand steps need to satisfy DOS criteria for currently active monitoring intervals, how the particular clinic is tracking overall on monitoring compliance, and patients who have satisfied billing criteria. The transmission metrics may be sorted by type of patient care modality as well as by clinic (e.g., based on a common clinic identifier associated with multiple transmissions), by site, by device type, and by date of service (e.g., via user inputs at one or more interactive graphical element such as drop down menus, text fields, selectable icons, slide bars, etc.) The transmission metrics may include one or more of an indication of a number of passed or extended DOSs, a number of patients with no DOS (including patients that have not opted into any patient care modality racking) a number of actions needed in 10 or less days, a number of actions needed in 11 or more days, and/or a number of monitoring intervals that have the DOS criteria satisfied and are waiting for the DOS to occur. Additionally, the transmission dashboard may provide patient filtering to generate a list of patients that is sorted by a number of days until the predicted DOS (e.g., end date), by a status (e.g., DOS criteria satisfied, docket report needs approval, no docket report, no DOS, no transmission, and/or DOS passed or extended) and/or by entering a patient name.

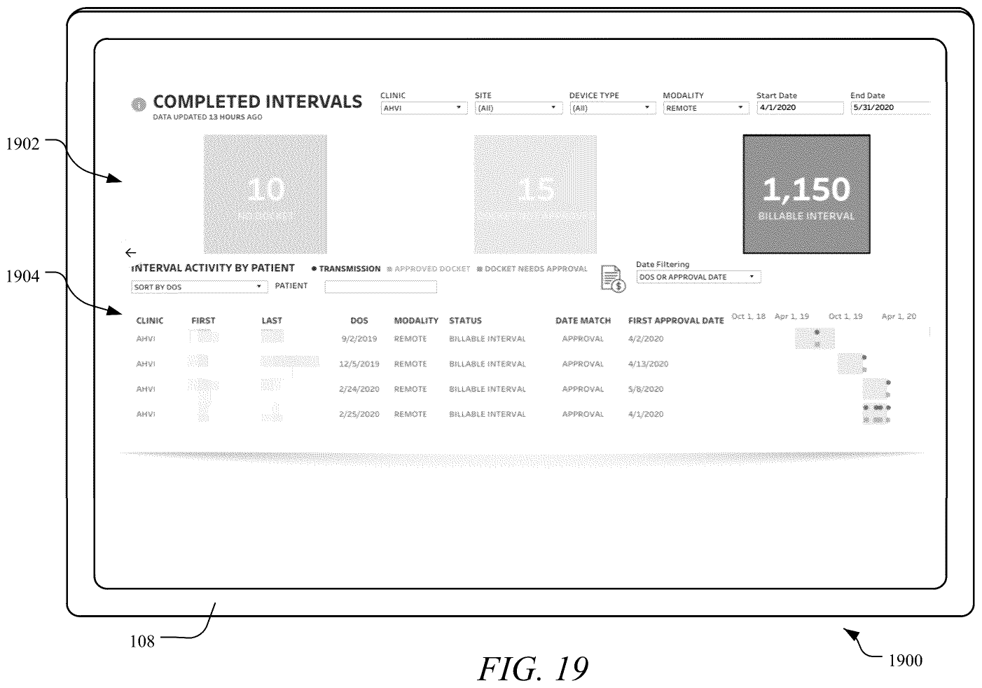

FIG. 19 illustrates a billing report dashboard 1900 that may be generated by the monitoring interval tracker 222. The billing report dashboard may present one or more transmission metrics related to DOS criteria, such as a number of monitoring intervals with no docket report, a number of monitoring intervals with a docket report needing approval, and/or a number of monitoring intervals that have the DOS criteria satisfied and, therefore, are billable monitoring intervals. The billing report dashboard 1900 may include a first section 1902 presenting one or more interactive filters for receiving user input on which the transmission metrics related to DOS criteria are based (e.g., a clinic, a site, a device type, a patient care modality, a start date, and/or an end day). The first section 1902 may present the transmission metrics related to DOS criteria as interactive indicators (e.g., selectable blocks), that, upon being selected, present additional information related to the transmission metric in a second section 1904 below the first section. For instance, upon receiving user input at the billable monitoring intervals indicator, the second section may present a list of monitoring intervals with additional information related to the monitoring intervals, such as a clinic name, a patient name, a DOS, a patient care modality, a status, a date match, and/or a first approval date. The billing report dashboard may present one or more interactive elements for, upon receiving an input, converting the transmission metrics into one or more billing reports (e.g., that are downloadable, sendable, and/or printable) to indicate that monitoring intervals for a patient are currently billable or what action is required to satisfy DOS criteria so that the monitoring intervals for the patient can become billable. Billing reports may be generated weekly or based on a user input selecting a frequency for generating billable reports. The data for the billing reports (e.g., the transmission metrics) may be generated as a downloadable spreadsheet. Billing reports may be downloadable reports that include a patient name, date of birth, medical record number (MRN), device type, device model, device implant date, start and end dates of the monitoring interval, opted into types of patient care modality (e.g., the in-office care modality 1204, the remote monitoring care modality 1206, and/or the heart failure care modality 1208), approving provider, approval date, professional and technical CPT codes, and/or ICD-10 code. In some instances, a billing report generator may take into account many of the variables discussed herein, and may check that the DOS criteria is satisfied in order for the patient name to appear on the billing report, such that only one billing report is generated per monitoring interval per patient, reducing an amount of time clinics spend tracking down such pertinent data. In some examples, recognizing a first approval date in a monitoring interval as satisfying DOS criteria (and not any subsequent approval dates) provides additional assurance of compliance to know that billing that billing requirements for that monitoring interval are satisfied. The billing report dashboard 1900 may output one or more reports (e.g., indicating the transmission metrics), such as the billing reports and/or patient care reports indicating practice and/or retroactive actions the clinic may take to ensure compliance (e.g., that the DOS criteria for the monitoring intervals is satisfied)

In some examples, operations performed by the monitoring interval tracker 222 to generate the monitoring interval visualizer 410, the transmissions dashboard 412, and/or the billing report dashboard may result in visualizing data trends, levels of completed and pending care, and workflow action in a graphical interface that can simplify complex data and parameters into potentially millions of unique combinations. Generating visualizations may also provide a “compliance to-do list’ describing for each type of patient care modality what actions need to be taken to satisfy DOS criteria for currently active monitoring intervals.

Various non-limiting example embodiments of the monitoring interval tracker 222, the monitoring interval visualizer 410, the transmission dashboards 412, and/or other components of the medical device platform 204 are discussed below:

In some instances, a method for managing a cardiac monitoring device may comprise: determining a monitoring interval associated with the cardiac monitoring device; presenting, at a display, a block representing the monitoring interval on one or more timelines corresponding to one or more patient care modes; accessing transmission data originating from the cardiac monitoring device; presenting, at the one or more timelines, one or more icons including a transmission icon representing a received date of the transmission data and a docket icon corresponding to the transmission data; receiving one or more inputs at least indicating an approval date of a docket report associated with the docket icon; and determining a date of service (DOS) for the transmission data based at least in part on the received date, the one or more patient care modes, and the approval date.

Moreover, the one or more timelines may comprise a plurality of timelines presented at the display simultaneously and extending horizontally, the plurality of timelines comprising: a first timeline corresponding to an in-office care mode of the one or more patient care modes; a second timeline corresponding to a heart failure care mode of the one or more patient care modes; a third timeline corresponding to a remote monitoring care mode of the one or more patient care modes; and a fourth timeline corresponding to transmissions from the cardiac monitoring device.

Moreover, the first timeline may represent monitoring intervals as one or more one-year blocks, the second timeline represents monitoring intervals as one or more 31-day blocks, and the third timeline represents monitoring intervals as 91-day blocks.

Moreover, receiving the one or more inputs may include: receiving a first input providing an impression for the docket report; and receiving a second input providing approval of the docket report; and the one or more icons may include a first indication of the impression and a second indication of the approval.

Moreover, the method may further comprise presenting, at the display, one or more input elements for enrolling a patient associated with the cardiac monitoring device, the one or more input elements including one or more of: a first selectable icon for indicating a particular patient care mode of the one or more patient care modes; a second selectable icon for indicating an interval extension rule; an first input field for indicating a length of the monitoring interval; and an second input field for indicating the date of service.

Moreover, the received date may occur after an end of the monitoring interval, and the method may further comprise: generating an extension to the monitoring interval based at least partly on the received date occurring after the end of the monitoring interval and based at least partly on an interval extension rule.

Moreover, the method may further comprise determining, based at least partly on the one or more patient care modes, that the interval extension rule extends the monitoring interval by day until an approval is received.

Moreover, the block may comprise a first block, and the method may further comprise determining, based at least partly on the one or more patient care modes, that the interval extension rule adds an extension monitoring interval represented by a second block having a same length as the first block representing the monitoring interval when the received date is outside the monitoring interval.

Moreover, the monitoring interval may comprise a first monitoring interval, the transmission data may comprise first transmission data, and the method may further comprise: presenting, at a side of the one or more timelines, one or more action needed icons indicating that at least one of: a second monitoring interval needs second transmission data; third transmission data corresponding to a heart failure care mode needs an impression; a patient associated with an in-office care mode needs a visit; or fourth transmission data corresponding to the heart failure care mode, the in-office care mode, or a remote monitoring care mode needs a docket report.

Moreover, the method may further comprise: receiving a selection of the docket icon; and presenting, at the display and below the one or more timelines, one or more docket reports associated with the transmission data at least partly in response to the selection of the docket icon.

In some instances, a method for managing a cardiac monitoring device may comprise: determining a plurality of monitoring intervals associated with the cardiac monitoring device; presenting, at a display, a series of blocks representing the plurality of monitoring intervals on a timeline, a block of the series of blocks having a length based on a patient care mode corresponding to the timeline; accessing transmission data originating from the cardiac monitoring device; presenting, at the timeline, one or more icons including a transmission icon representing a received date of the transmission data and a docket icon corresponding to the transmission data; and determining a date of service (DOS) for the transmission data based at least in part on the received date, the patient care mode, and an approval date.

Moreover, the method may further comprise: receiving a selection of the block representing a monitoring interval of the plurality of monitoring intervals; and presenting, at least partly in response to the selection of the block, an interval report, the monitoring interval being: a completed monitoring interval and the interval report indicating historical information related to the completed monitoring interval; or an active monitoring interval and the interval report indicating needed approvals that are pending.

Moreover, the method may further comprise presenting, at the display, one or more input elements for generating a transmission report for a clinic, the one or more input elements corresponding to a care mode, a clinic site, or days-to-DOS.

Moreover, the method may further comprise: receiving input at the one or more input elements; and presenting, at the display and at least partly in response to the input, one or more transmission related metrics representing aggregated transmission data for the clinic.

Moreover, the one or more transmission related metrics may include one or more of: a number of monitoring intervals waiting to receive transmissions; a number of transmissions waiting to receive docket reports; a number of docket reports waiting to receive approval; a number of monitoring intervals that have been completed; a number of patients that opted into a patient care mode; a number of patients that opted out of the patient care mode; and a number of monitoring intervals that need DOSs.

Moreover, the method may further comprise receiving a selection of a monitoring report icon presented at a sidebar on the display, presenting the one or more input elements for generating the transmission report being at least partly in response to the selection of the monitoring report icon.

Moreover, the method may further comprise: presenting, at the display, a DOS analytics dashboard including one or more filter input elements corresponding to a clinic, a clinic site, a care mode, a cardiac monitoring device type, a date of service, a number of days to DOS, a monitoring interval status, or a patient name; receiving input at the one or more filter input elements; and presenting, at the display and based at least partly on receiving the input at the one or more filter input elements, one or more DOS related metrics representing aggregated DOS data.

Moreover, the DOS related metrics may include one or more of: a number of monitoring intervals that have passed the DOS or lack the DOS; a number of monitoring intervals needing action in 10 days or less; a number of monitoring intervals needing action in 11 or more days; and a number of monitoring intervals that have completed DOS criteria.

Moreover, the method may further comprise: presenting, at the display, a billing report dashboard for completed monitoring intervals that includes one or more filter input elements corresponding to a clinic, a clinic site, a cardiac monitoring device type, a care mode, a date range, DOSs, or a patient name; receiving input at the one or more filter input elements; and presenting, at the display and based at least partly on receiving the input at the one or more filter input elements, one or more of: an indication of a number of monitoring intervals needing a docket report; a number of docket reports needing approval; or a number of monitoring intervals ready for billing.

In some instances, a method for managing a cardiac monitoring device may comprise: determining a first monitoring interval associated with the cardiac monitoring device, the first monitoring interval having a first length corresponding to a first patient care mode; determining a second monitoring interval associated with the patient, the second monitoring interval having a second length corresponding to a second patient care mode; presenting, at a display, the first monitoring interval as a first block on a first timeline corresponding to the first patient care mode, and simultaneously presenting the second monitoring interval as a second block on a second timeline corresponding to the second patient care mode; accessing transmission data originating from the cardiac monitoring device; presenting, at a third timeline corresponding to transmissions, one or more transmission icons representing one or more received dates of the transmission data; presenting, at the first timeline a first docket icon corresponding to the transmission data; presenting, at the second timeline, a second docket icon corresponding to the transmission data; receiving one or more inputs at least indicating an approval date of a docket report associated with the first docket icon or the second docket icon; and determining a date of service (DOS) for the transmission data based at least in part on the one or more received date and the approval date.

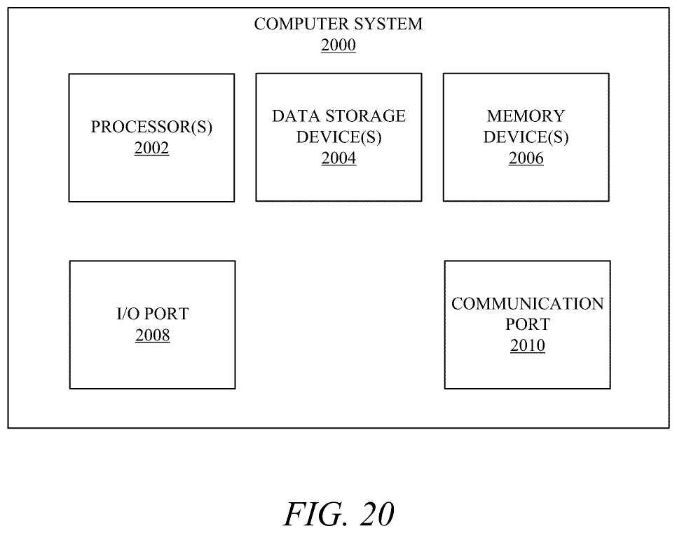

Referring to FIG. 20, a detailed description of an example computing system 2000 having one or more computing units that may implement various systems and methods discussed herein is provided. The computing system 2000 may be applicable to the medical device manager 102 and/or the user computing device 108 and other computing or network devices. It will be appreciated that specific implementations of these devices may be of differing possible specific computing architectures not all of which are specifically discussed herein but will be understood by those of ordinary skill in the art.

The computer system 2000 may be a computing system is capable of executing a computer program product to execute a computer process. Data and program files may be input to the computer system 2000, which reads the files and executes the programs therein. Some of the elements of the computer system 2000 are shown in FIG. 20, including one or more hardware processors 2002, one or more data storage devices 2004, one or more memory devices 2006, and/or one or more ports 2008-2010. Additionally, other elements that will be recognized by those skilled in the art may be included in the computing system 2000 but are not explicitly depicted in FIG. 20 or discussed further herein. Various elements of the computer system 2000 may communicate with one another by way of one or more communication buses, point-to-point communication paths, or other communication means not explicitly depicted in FIG. 20.

The processor 2002 may include, for example, a central processing unit (CPU), a microprocessor, a microcontroller, a digital signal processor (DSP), and/or one or more internal levels of cache. There may be one or more processors 2002, such that the processor 2002 comprises a single central-processing unit, or a plurality of processing units capable of executing instructions and performing operations in parallel with each other, commonly referred to as a parallel processing environment.

The computer system 2000 may be a conventional computer, a distributed computer, or any other type of computer, such as one or more external computers made available via a cloud computing architecture. The presently described technology is optionally implemented in software stored on the data stored device(s) 2004, stored on the memory device(s) 2006, and/or communicated via one or more of the ports 2008-2010, thereby transforming the computer system 2000 in FIG. 20 to a special purpose machine for implementing the operations described herein. Examples of the computer system 2000 include personal computers, terminals, workstations, mobile phones, tablets, laptops, personal computers, multimedia consoles, gaming consoles, set top boxes, and the like.

The one or more data storage devices 2004 may include any non-volatile data storage device capable of storing data generated or employed within the computing system 2000, such as computer executable instructions for performing a computer process, which may include instructions of both application programs and an operating system (OS) that manages the various components of the computing system 2000. The data storage devices 2004 may include, without limitation, magnetic disk drives, optical disk drives, solid state drives (SSDs), flash drives, and the like. The data storage devices 2004 may include removable data storage media, non-removable data storage media, and/or external storage devices made available via a wired or wireless network architecture with such computer program products, including one or more database management products, web server products, application server products, and/or other additional software components. Examples of removable data storage media include Compact Disc Read-Only Memory (CD-ROM), Digital Versatile Disc Read-Only Memory (DVD-ROM), magneto-optical disks, flash drives, and the like. Examples of non-removable data storage media include internal magnetic hard disks, SSDs, and the like. The one or more memory devices 2006 may include volatile memory (e.g., dynamic random access memory (DRAM), static random access memory (SRAM), etc.) and/or non-volatile memory (e.g., read-only memory (ROM), flash memory, etc.).

Computer program products containing mechanisms to effectuate the systems and methods in accordance with the presently described technology may reside in the data storage devices 2004 and/or the memory devices 2006, which may be referred to as machine-readable media. It will be appreciated that machine-readable media may include any tangible non-transitory medium that is capable of storing or encoding instructions to perform any one or more of the operations of the present disclosure for execution by a machine or that is capable of storing or encoding data structures and/or modules utilized by or associated with such instructions. Machine-readable media may include a single medium or multiple media (e.g., a centralized or distributed database, and/or associated caches and servers) that store the one or more executable instructions or data structures.

In some implementations, the computer system 2000 includes one or more ports, such as an input/output (I/O) port 2008 and a communication port 2010, for communicating with other computing, network, or vehicle devices. It will be appreciated that the ports 2008-2010 may be combined or separate and that more or fewer ports may be included in the computer system 2000.

The I/O port 2008 may be connected to an I/O device, or other device, by which information is input to or output from the computing system 2000. Such I/O devices may include, without limitation, one or more input devices, output devices, and/or environment transducer devices.

In one implementation, the input devices convert a human-generated signal, such as, human voice, physical movement, physical touch or pressure, and/or the like, into electrical signals as input data into the computing system 2000 via the I/O port 2008. Similarly, the output devices may convert electrical signals received from computing system 2000 via the I/O port 2008 into signals that may be sensed as output by a human, such as sound, light, and/or touch. The input device may be an alphanumeric input device, including alphanumeric and other keys for communicating information and/or command selections to the processor 2002 via the I/O port 2008. The input device may be another type of user input device including, but not limited to: direction and selection control devices, such as a mouse, a trackball, cursor direction keys, a joystick, and/or a wheel; one or more sensors, such as a camera, a microphone, a positional sensor, an orientation sensor, a gravitational sensor, an inertial sensor, and/or an accelerometer; and/or a touch-sensitive display screen (“touchscreen”). The output devices may include, without limitation, a display, a touchscreen, a speaker, a tactile and/or haptic output device, and/or the like. In some implementations, the input device and the output device may be the same device, for example, in the case of a touchscreen.

The environment transducer devices convert one form of energy or signal into another for input into or output from the computing system 2000 via the I/O port 2008. For example, an electrical signal generated within the computing system 2000 may be converted to another type of signal, and/or vice-versa. In one implementation, the environment transducer devices sense characteristics or aspects of an environment local to or remote from the computing device 2000, such as, light, sound, temperature, pressure, magnetic field, electric field, chemical properties, physical movement, orientation, acceleration, gravity, and/or the like. Further, the environment transducer devices may generate signals to impose some effect on the environment either local to or remote from the example computing device 2000, such as, physical movement of some object (e.g., a mechanical actuator), heating or cooling of a substance, adding a chemical substance, and/or the like.

In one implementation, a communication port 2010 is connected to a network by way of which the computer system 2000 may receive network data useful in executing the methods and systems set out herein as well as transmitting information and network configuration changes determined thereby. Stated differently, the communication port 2010 connects the computer system 2000 to one or more communication interface devices configured to transmit and/or receive information between the computing system 2000 and other devices by way of one or more wired or wireless communication networks or connections. Examples of such networks or connections include, without limitation, Universal Serial Bus (USB), Ethernet, Wi-Fi, Bluetooth®, Near Field Communication (NFC), Long-Term Evolution (LTE), and so on. One or more such communication interface devices may be utilized via the communication port 2010 to communicate one or more other machines, either directly over a point-to-point communication path, over a wide area network (WAN) (e.g., the Internet), over a local area network (LAN), over a cellular (e.g., third generation (3G) or fourth generation (4G)) network, or over another communication means. Further, the communication port 2010 may communicate with an antenna or other link for electromagnetic signal transmission and/or reception.

The system set forth in FIG. 20 is but one possible example of a computer system that may employ or be configured in accordance with aspects of the present disclosure. It will be appreciated that other non-transitory tangible computer-readable storage media storing computer-executable instructions for implementing the presently disclosed technology on a computing system may be utilized.

In the present disclosure, the methods disclosed may be implemented as sets of instructions or software readable by a device. Further, it is understood that the specific order or hierarchy of steps in the methods disclosed are instances of example approaches. Based upon design preferences, it is understood that the specific order or hierarchy of steps in the method can be rearranged while remaining within the disclosed subject matter. The accompanying method claims present elements of the various steps in a sample order, and are not necessarily meant to be limited to the specific order or hierarchy presented.

The described disclosure may be provided as a computer program product, or software, that may include a non-transitory machine-readable medium having stored thereon instructions, which may be used to program a computer system (or other electronic devices) to perform a process according to the present disclosure. A machine-readable medium includes any mechanism for storing information in a form (e.g., software, processing application) readable by a machine (e.g., a computer). The machine-readable medium may include, but is not limited to, magnetic storage medium, optical storage medium; magneto-optical storage medium, read only memory (ROM); random access memory (RAM); erasable programmable memory (e.g., EPROM and EEPROM); flash memory; or other types of medium suitable for storing electronic instructions.

While the present disclosure has been described with reference to various implementations, it will be understood that these implementations are illustrative and that the scope of the present disclosure is not limited to them. Many variations, modifications, additions, and improvements are possible. More generally, implementations in accordance with the present disclosure have been described in the context of particular implementations. Functionality may be separated or combined in blocks differently in various implementations of the disclosure or described with different terminology. These and other variations, modifications, additions, and improvements may fall within the scope of the disclosure as defined in the claims that follow.

What is claimed is:

1. A method for managing a cardiac monitoring device, the method comprising:
generating a monitoring interval associated with the cardiac monitoring device, the monitoring interval having an end date;
accessing transmission data having a received date and originating from the cardiac monitoring device;
generating a docket report for the transmission data;
accessing, from a rules database, an interval extension rule; and
generating, according to the interval extension rule, a monitoring interval extension having an extended end date based at least partly on whether the received date is later than the end date.

2. The method of claim 1 further comprising:
calculating whether the received date is later than the end date includes determining, upon an occurrence of the end date, that the transmission data is yet to be received; and
generating the monitoring interval extension includes generating day-by-day extensions to the monitoring interval until the transmission data is received on the extended end date.

3. The method of claim 1 further comprising:
receiving an approval timestamp corresponding to the docket report.

4. The method of claim 3, wherein the approval timestamp corresponds to an approval date for the docket report.

5. The method of claim 4 further comprising:
calculating whether the approval date is later than the end date includes determining, upon an occurrence of the end date, that the approval timestamp is yet to be received; and
generating the monitoring interval extension includes generating day-by-day extensions to the monitoring interval until the approval timestamp occurs on the extended end date.

6. The method of claim 5, wherein the monitoring interval comprises a first monitoring interval and the end date comprises a first end date, and the method further comprises generating, in response to the approval timestamp occurring on the extended end date, a second monitoring interval having a second end date.

7. The method of claim 6, wherein the docket report is a first docket report, the approval timestamp is a first approval timestamp, and the method further comprises:
generating a second docket report for the transmission data;
receiving a second approval timestamp indicating an approval date later than the extended end date and prior to the second end date; and
determining that the second approval timestamp corresponds to the second docket report.

8. A method for managing a cardiac monitoring device, the method comprising:
generating a first monitoring interval associated with the cardiac monitoring device, the first monitoring interval having a first end date;
generating a second monitoring interval associated with the cardiac monitoring device, the second monitoring interval having a second end date, the second end date being different than the first end date;
accessing transmission data having a received date and originating from the cardiac monitoring device;
generating, for the transmission data, a first docket report;
generating, for the transmission data, a second docket report;
accessing, from a rules database, an interval extension rule; and
determining, according to the interval extension rule, whether to generate a monitoring interval extension having an extended end date based at least partly on whether the received date is later than the first end date.

9. The method of claim 8 further comprising:
receiving an approval timestamp after the received date and indicating an approval date corresponding to only one the first docket report or the second docket report.

10. The method of claim 9, wherein the approval timestamp corresponds to an approval date corresponding to the first docket report or the second docket report.

11. The method of claim 10 further comprising:
calculating whether the approval date is later than the first end date includes determining, upon an occurrence of the first end date, that the approval timestamp is yet to be received; and
generating the monitoring interval extension includes generating day-by-day extensions to the first monitoring interval until the approval timestamp occurs on the extended end date.

12. The method of claim 11, further comprising generating, in response to the approval timestamp occurring on the extended end date, a third monitoring interval running subsequently to the first monitoring interval and having a third end date.

13. The method of claim 12, wherein the approval timestamp is a first approval timestamp, the approval date is a first approval date corresponding to the first docket report, and the method further comprises:
receiving, prior to the third end date of the third monitoring interval, a second approval timestamp indicating a second approval date; and
determining that the second approval date corresponds to the second docket report.

14. A method for managing a cardiac monitoring device, the method comprising:
generating a plurality of monitoring intervals, running concurrently, and associated with the cardiac monitoring device, a first monitoring interval of the plurality of monitoring intervals having a first end date and a second monitoring interval of the plurality of monitoring intervals having a second end date that is different than the first end date;
accessing transmission data having a received date prior to the first end date and originating from the cardiac monitoring device;
generating, for the transmission data, a docket report associated with the first monitoring interval or the second monitoring interval;
accessing, from a rules database, an interval extension rule; and
generating, according to the interval extension rule, a monitoring interval extension having an extended end date based at least partly on the received date being received later than the first end date.

15. The method of claim 14 further comprising:
receiving an approval timestamp corresponding to the docket report.

16. The method of claim 15, wherein the approval timestamp corresponds to an approval date for the docket report.

17. The method of claim 16, wherein:
calculating that the approval date is later than the first end date includes determining, upon an occurrence of the first end date, that the approval timestamp is yet to be received; and
generating the monitoring interval extension includes generating day-by-day extensions to the first monitoring interval until the approval timestamp occurs on the extended end date.

18. The method of claim 16, further comprising generating, in response to the approval timestamp occurring on the extended end date, a third monitoring interval having a third end date.

19. The method of claim 15, further comprising:
generating a graphical representation of the plurality of monitoring intervals as interactive blocks on a plurality of simultaneously presented timelines.

20. The method of claim 19, the plurality of simultaneously presented timelines including:
a first timeline including the first monitoring interval with a 31-day length;
a second timeline including the second monitoring interval with a 91-day length; and
a third timeline including a third monitoring interval with a one-year length.
